(19) United States
(12) Patent Application Publication
(10) Pub. No.: US 2022/0346917 A1
(43) Pub. Date: Nov. 3, 2022

(54) CRANIAL RESTRUCTURING DEVICES

(71) Applicant: Shailen Patel, Harrow, Greater London (GB)

(72) Inventors: Shailen PATEL, Harrow (GB); Bill HOWES, London (GB)

(73) Assignee: Shailen PATEL, Harrow, Greater London (GB)

(21) Appl. No.: 17/621,089

(22) PCT Filed: Jun. 19, 2020
(86) PCT No.: PCT/EP2020/067216

§ 371 Date: Dec. 20, 2021

(30) Foreign Application Priority Data
Jun. 20, 2019 (GB) 1908862.4

(51) Int. Cl. A61C 7/10; A61B 17/66; A61C 7/00

(52) U.S. Cl. CPC A61C 7/10; A61B 17/666; A61C 7/008; A61B 2017/00539

(57) ABSTRACT

A device for cranial restructuring, by the expansion, compression, or flexure of a cranial structure located between first and second anchor points, includes: a force generator configured to generate force; a first anchor attachment to the first anchor point; a second anchor attachment to a second anchor point; a hydraulic force transmitting structure, connected to the force generator, to transmit the generated force to the anchors thereby putting the cranial structure in tension, compression or flexure. Another device for cranial restructuring includes: a head support, having a head-receiving portion; a force generator to generate force which is periodic; a first anchor attachment to the first anchor point; a force transmitting structure, connected to the force generator, which transmits the periodic force to the anchor in a restructuring direction; wherein the head support includes: a restriction to prevent or restrict movement of the user's head when the periodic force is applied.

The present invention relates to a device for cranial and/or orthodontic restructuring achieved primarily by the action of a periodic force.

### BACKGROUND TO THE INVENTION

It is well known that dental devices or systems such as braces, intra-oral appliances, retainers etc. may be used to reposition teeth and modify the dental arch of a user. These work by applying a constant, static force to the teeth to which they are attached. More accurately, as the teeth change position as a result of the constant force being applied to them, the force experienced by each tooth decays with time.

In many cases, devices must be worn 24 hours a day such as with fixed braces, and constantly exert a force on the user's teeth, in order to have any meaningful effect. This can become uncomfortable for the user. A similar situation can arise when trying to correct a user's facial bone structure. This often requires unwieldy bracing structures e.g. head gear which may be removable or fixed in the form of cranial distraction apparatus, often requiring more than 12 hours of wear time or constant usage in the case of fixed devices.

### SUMMARY OF THE INVENTION

Broadly speaking, the present invention aims to address this problem by providing a device which is configured to apply a periodic force to the bones, teeth, or other parts of the body, in order to remodel their structure, for example by expansion, compression or protraction. As will be set out below, in some cases, the periodic force which is applied is superposed onto a static force for added efficacy.

A first aspect of the invention relates primarily to devices configured to perform restructuring of the maxilla, mandible, dental arch, or palate — but it will be appreciated that devices according to embodiments of the first aspect of the invention may be used for other types of cranial restructuring. More specifically, a first aspect of the present invention achieves this in the provision of a device for cranial restructuring, by the expansion, compression, or flexure of a cranial structure located between a first anchor point and a second anchor point, the device including:
a force generator configured to generate a periodic force;
a first anchor for attachment to the first anchor point;
a second anchor for attachment to a second anchor point;
a force transmitting structure, connected to the force generator, and configured to transmit the periodic force to the first anchor and the second anchor thereby putting the cranial structure in at least one of: tension, compression or flexure. It is recognised that there may be more than two anchors and that an anchor point may constitute a grouping of structures.

It has been shown that the application of a dynamic force to bone structure results in greater bone growth compared to a static force. A combination of static and dynamic forces has also been shown to accelerate tooth movement. This is advantageous as it reduces treatment time minimizing the amount of pain/discomfort experienced by the user. It also minimizes or removes the need to wear a static brace or oral appliance/retainer for a long time.

Throughout this application, it is appreciated that forces applied to teeth can also lead to compression and tension of intermediary structures such as the periodontal ligament and under differing loading regimens the effect on tooth movement and bone remodelling will vary. It is also appreciated that cranial structures and sutures may be subject to varying amounts of compression and/or tension depending on the loading regimen.

It is preferred that the force generator is an external force generator. In other words, in use, the force generator is preferably located or locatable outside the user's body. Here “periodic force” may refer to a cyclic force (i.e. a force which is periodic and has a constant frequency or period), but it should also be understood to cover quasi-periodic forces, in which for example, the frequency or time period varies. Alternatively put, the force may be variable force with a variable time period. For cyclic frequencies, it is preferable that the frequency is in the range of 1 to 400 Hz, and in embodiments in which the frequency varies (i.e. quasi-periodic forces), the frequency preferably varies between 1 Hz and 400 Hz. It is preferred that the waveform of the periodic or quasi-periodic force is one of: triangular, sinusoidal, sawtooth, spiked, or square. The force may also be any superposition of these. Other periodic waveforms should be understood to be covered by this definition.

The periodic force is preferably a unidirectional force. In other words, the action of the periodic force is preferably not a “push-pull” action, but rather either a push or pull action which varies periodically (or quasi-periodically) with time. In this way, the force which is applied to the primary cranial structure acts to give rise to restructuring only in a single direction. The force generator may be configured to produce forces other than a periodic force, for example, a continuously varying force, or a constant force—or combinations thereof. Specifically, the profile of the force generated by the force generator may include periodic and non-periodic regions, as well as regions where no force is applied. For example, in some embodiments, the force generator may be configured to generate a force having a profile with alternating periodic and non-periodic regions. In some embodiments, the non-periodic regions may include a constant force, and in other regions, the non-periodic regions may include a force which is increasing/decreasing, linearly or otherwise. In preferred embodiments, the periodic/quasi-periodic part of the force preferably oscillates around a non-zero force. Preferably, when the force oscillates around a non-zero force, the minima of the oscillations do not go below zero. In this way, it is possible to ensure that a force is always being applied to the primary cranial structure in question. This is in contrast to a force which oscillates around a zero point, where for around half the time, there may be no force applied. In some embodiments, the force characteristics (e.g. duration, amplitude) may be adjusted based on continuous or intermittent input from a data processor arranged to receive input from a measurement device such as for example: a pressure gauge, strain gauge, a device measuring the displacement of the cranial structure or other bodily structure. Other forms of input are also covered e.g. Electrocardiogram (ECG), Electroencephalogram (EEG) and Electromyography (EMG). In this way, the device may operate as a feedback system. In some cases the feedback may be from the operation of one embodiment informing the activity of one or more other devices. A constant rate of distraction can be achieved using hydraulics in conjunction with a displacement feedback mechanism.

It should be noted that throughout this application, we refer to “constant forces” or “static forces”. When a constant force is applied to e.g. a tooth, or other cranial structure, over time that force will act to cause movement of the structure in the direction of the constant force. The force at contact is preferably constant.

If the force is provided by e.g. a tensioned wire or an inflatable structure, then the tension in the wire (or inflatable structure) will decrease as the structure moves, i.e. the static force will decrease very slowly over time. Throughout this application, these forces are still referred to as “static forces” or “constant forces” despite the fact that they change gradually over time. Throughout this application, the terms “constant force” and “static force” should still be considered to cover forces which only vary a negligible amount over the order of the timescale of one period of the periodic force or not at all. A “true” constant force may be generated by an actuator with a feedback loop. As the structure moves the pressure in the system falls and is corrected to its initial value thus maintaining a constant force. The surface area of contact between the anchor point and anchor may change after a correction or series of corrections.

In some cases, there may be two means by which force is applied to the first anchor or the second anchor. Specifically, a first component may apply a static force to the first anchor and/or the second anchor, and a second component may apply the periodic force to the first anchor and/or the second anchor. In this way, a constant “background” force may be applied, and the periodic force generated by the force generator may be superposed over that. It is preferred that the force provided by the first component is adjustable, for example by means of a screw. The first component may be in the form of a well-known oral appliance such as a static brace, retainer or rapid maxillary expander. In such cases, when the user wears the device, the cranial structure to be restructured undergoes a constant, static tension or extension force. For example, if a user in need of maxillary dental arch expansion wears such a device, that device may apply a constant force in a laterally-outward direction to the upper teeth, that force on the teeth acting to put the upper palate in tension, and the upper jaw in flexure, thus giving rise to gradual lateral maxillary expansion.

When the device includes a static force generating component, the force generator need then not generate a periodic force having such a large magnitude, because the periodic component of the force is then superposed onto the static component of the force provided by the static component. Alternatively the force generator could generate all or some of the static force as well the periodic component. The static component is preferably shaped to expose the first anchor and the second anchor to the first anchor point and the second anchor point respectively, in order to ensure that the anchors can attach to the anchor points, for efficient force transmission. For example, the static component may include holes or windows. Alternatively, the static component may include means for connecting it to the remainder of the device, in use, to ensure that the components of the forces add constructively, rather than, for example acting to cancel each other out. Embodiments of the invention in which the device includes a static component ensure that the force resulting from the superposition of the static and periodic components of the force is always positive, i.e. acting in the correct direction for the desired restructuring to take place.

Embodiments of the present invention are adapted to perform cranial restructuring by effecting either expansion, compression, or flexure of cranial structures. For example, in some embodiments, the device may be used for mandibular or maxillary expansion, in which the mandible/maxilla is expanded laterally as well as anteriorly. Alternatively, the device may be used for maxillary or mandibular protraction, in which the mandible/maxilla is brought forward, e.g. of the bone in question. Protraction may also stimulate growth by subjecting the bone to strain. In some embodiments other movements such as retraction of teeth or the maxilla can be achieved. This also includes medial or more generally an inward movement of the teeth. Movement of a tooth or teeth along or about any axis or combinations thereof such as rotation may also be possible in some embodiments of the invention. It is noted that in some applications of the invention, the desired effect is the movement of two cranial structures (e.g. maxillary expansion, where the movement of both the left and right side of the maxilla is desirable, i.e. bony displacement whereby the left side and the right side of the maxilla are separated by disarticulation of adjoining sutures or simply separated further where no or insignificant fusion of sutures has taken place), and in other applications, the desired effect is the movement of one cranial structure (e.g. maxillary protraction, where the desired outcome is moving forward of the maxilla). However, the mode of operation in both of these cases is effectively identical, since both are achieved by the expansion of some cranial structure in order to increase the distance between two fixed locations on the cranium. The same applies to compressive, and flexural actions. This application covers various different uses of the present invention, in addition to those mentioned in this paragraph. It can be recognised that asymmetric treatment of the structure in question can be accomplished e.g. one side of the maxilla with or without separation of sutures

The device of the present invention works by transmitting a first force to the first anchor, and a second force to the second anchor, wherein the first force is in the opposite direction to the second force, or substantially so. In embodiments where the first force or the second force are applied over a large area (e.g. between a head plate and the back of the head), the net effect of the first force is opposite to the net effect of the second force, or substantially so. In some embodiments, the force transmitting structure is configured to apply the first (periodic) force directly to the first anchor only. In those embodiments, the first force is transmitted to the second anchor via the cranial structure, which experiences the second force as a reaction force, having the same magnitude (or substantially the same magnitude) as the first force, but in the opposite direction. As the skilled person is well aware, the application of two forces in opposing directions to the cranial structure, via the two anchor points, gives rise to the tension or compression force in the cranial structure, which then effects the desired cranial restructuring. In some embodiments, e.g. when the cranial structure is not located between on the straight line connecting the first anchor point and the second anchor point, the opposing first and second forces instead may act to give rise to flexure of the cranial structure. This applies equally well to cases in which the periodic force is applied to the second anchor only, but for conciseness, we will not repeat this description here.

In other embodiments, the force transmitting structure is configured to apply the periodic force directly to the first anchor and the second anchor. Specifically, the force transmitting structure is configured to apply the first force directly to the first anchor and the second force directly to the second anchor. As above, it is preferred that the first force and the second force are equal in magnitude and opposite in direction, in order to generate tension or compression in the cranial structure located between the first anchor point and the second anchor point.

Above, we discuss the mechanics of the forces which are transmitted to the first anchor and the second anchor. Now we discuss the nature of those forces, and their transmission to the first anchor and the second anchor by the force transmitting structure. In some embodiments, the force generator includes a motor such as a stepper motor which is configured to generate rotary motion. In such cases, the force generator preferably further includes means for converting rotary motion into reciprocating motion. These means may include a crank, screw thread or a cam. The skilled person is aware that there exists a wealth of other components which could be used to effect this conversion. As discussed, the force generator is preferably an external force generator, and as will become apparent, the first anchor point and second anchor point are generally located inside some cranial structure of the user, be it e.g. the mouth or the nose. Accordingly, the force transmitting structure is preferably configured to transmit the periodic force from outside the user's body to a location inside the user's body. Specifically, in preferred embodiments of the present invention, the force transmitting structure is configured to direct the force in an anterior-posterior direction, i.e. in a forward-backward direction with respect to a user's body. It is noted that in some embodiments, the periodic force may be generated in this direction, in which case the force transmitting structure has only to maintain that direction, but in alternative embodiments, the force may be generated in, e.g. a superior-inferior direction, and the force transmitting structure includes a mechanism for changing the direction of the force. The force generator may be located within a housing, the housing being configured for attachment to a harness. In some embodiments, the harness may be a chest harness.

The force transmitting structure may include one or more inextensible and incompressible wires configured to transmit the generated periodic force as tension. However, in preferred embodiments of the present invention, the force transmitting structure is a hydraulic force transmitting structure, which exploits the incompressible nature of fluids such as water or oils to transmit the force generated by the force generator as a compression force. The hydraulic force transmitting structure preferably includes one or more inflatable/collapsible structure, e.g. balloons or bellows, which can be inflated with fluid in order to transmit the periodic force to the first anchor or the second anchor. Specific embodiments of the present invention employing hydraulic transmitting structure will be described later on in this application. Hydraulic force transmitting structures are particularly effective, because they have been shown to reduce potential mechanical system whiplash. In other embodiments, the force transmission may include or be in the form of pneumatic or piezoelectric force transmitting structure.

In some embodiments of the present invention, the device may be modular. In other words, any or all of the force generator, force transmitting structure, first anchor and second anchor may be removable from the device so that they can be replaced with alternative components. In other embodiments, part of the device may be fixed or configured to be fixed to the cranial structure of a user, and connectable to the remainder of the device. For instance, at least one of the force transmitting structure, the first anchor, and the second anchor may be fixed or fixable to a cranial structure of the user, and connectable to the force generator. A modular structure of this kind is advantageous because it means that the fixed structure can be more securely fixed, e.g. surgically implanted or attached, which would lead to a more efficient transfer of force from the force generator to the cranial structure to be remodelled or restructured.

A modular structure is advantageous, for example when no force is required to be generated, the force transmitter and force generator may be detachable and in some embodiments the force may be self-maintained or exhausted. Thus the dwelling structure(s) remains as compact and unobtrusive as possible, improving patient comfort and minimising complications such as entanglement and other forms of accidental injury.

A modular hydraulic force transmitting structure is particularly advantageous because in some embodiments it may enable a single hydraulic force generator to be removably connected with multiple different portions of the force transmitting structure in order to adjust, individually, the force that is being applied by each of the different portions. Again this refers to a valvular or other form of detachable but self-sealing/sealing connection such as a one way or reversible valve. The valve may be proximal to the intra-oral appliance either within the body of the device or extending shortly from it. If the tubing leading from the mouth is not obtrusive the valve may be located closer to the hydraulic force generator. The valve allows for the force transmitter to be separable at any point along its length. The force transmitter maybe obtrusive when the device is not in use and can be detached from its connections, as well as being separable itself, by means of a self-sealing valve. This is not only convenient but reduces the risk of iatrogenic injury. The self-sealing valve allows the pressure in the system to be maintained. This pressure generates our fixed force which decays over time much like our wire under tension. Advantageously the system can be ‘reset’ several times a day (intermittent) such that the pressure and thus force on the teeth is maintained. Alternatively, as the cranial structure is displaced or remodelled, and thus the effective force/pressure on the structure falls, the device may be configured to increase the force provided by the force generator in order to ensure that a constant force is maintained on the cranial structure. According to an exemplary embodiment, the self-sealing valve may be configured to allow a hydraulic fluid carrying hose, or catheter, connectable and dis-connectable to a first hydraulic force transmitting structure portion. A first end of the hose may be fluidly connected to the hydraulic force generator and a second end of the hose may be configured to be reversibly connectable to the first hydraulic force transmitting structure portion. The hose may be further reversibly connected to a second hydraulic force transmitting structure portion. The self-sealing valve may be configured, when the hose is disconnected, to maintain the pressure of the hydraulic fluid in force transmitting structure portions. The modularity of the device components means that a single hydraulic force generator can be used to adjust the force that is applied to the user's cranial structure by a plurality of force transmitting structure portions. In this way, the modularity of the device removes the need to operate multiple hydraulic force generators, which thereby reduces the cost of the system. In situations where a device is required to apply simultaneous and differing force characteristics, then a number of hydraulic force generators may be employed. The hydraulic force generators may be separate units which can be connected together or provided within a single housing/single enclosed unit.

The above disclosure of the invention is general, and not linked to the application of the periodic force to specific cranial structures. Now, we turn to some specific applications of the device.

In some embodiments, the device is configured to perform maxillary or mandibular expansion, which may refer to widening of the dental arch due to tooth movement, or widening due to displacement of a bony section, which may exist naturally, be created by separation of sutures through use of a device or surgically. In this case, the first force and the second force give rise to outward flexure of the jaw in question, as well as expansion of the upper/lower surfaces of the mouth, i.e. making the mandible or maxilla less curved. It is recognised, that a structure being configured to receive force from such a device (either directly or indirectly) may experience that force as either compression or tension. There are a number of different ways that maxillary expansion can be achieved using devices of the present invention. In some embodiments, the first anchor point and/or the second anchor point may be teeth. For example, the first anchor and/or the second anchor may include wires, bands, or other orthodontic fixations configured to wrap around the teeth, or plates/wires which are configured to abut or engage with the inner or outer surfaces of the teeth. In embodiments in which the first anchor point and the second anchor point are a first tooth and a second tooth respectively, it is preferred that the first tooth is symmetrically opposite the second tooth in the user's mouth. In some embodiments, the first anchor and/or the second anchor may include a moulded plate which is shaped to conform to the inner surfaces of the tooth/teeth of a user in order to provide a snug fit, for more efficient force transfer. Alternative arrangements of the anchors may be employed depending of the shape of the dental arch and desired clinical outcome. In some embodiments, the first anchor and/or second anchor may be a combination of the features set out above. In other embodiments, the first anchor point and/or the second anchor point may be a location on the soft tissue of a user's mouth. In such embodiments, the first and/or second anchor may include a screw or equivalent fastener which is configured to contact bone or soft tissue directly. By contacting the tissue more directly, more efficient transfer of force is enabled.

According to an exemplary embodiment, the device may be in the form of, or include an expansion mechanism which may be configured to perform maxillary or mandibular expansion. The expansion mechanism may be configurable to be arranged at a substantially central position on the upper surface of a user's mouth. The expansion mechanism may comprise a central component, a hydraulic component, a channel component and an attachment component. According to an exemplary embodiment, the central component may be adjusted by turning the threaded portion, relative to the channel component, such that it applies a constant force to a patient's cranial structure, via the attachment component. This force defines a background static force. Then, in use, the hydraulic component is periodically inflated and deflated using an external hydraulic force generator. When the hydraulic component is inflated, it exerts an additional force onto the user's cranial structure via the attachment component. The additional force may be a periodic force. When this takes place simultaneously, the region of the user's cranial structure which is arranged between the two attachment components experiences an extension force which promotes maxillary expansion.

The central component may be configured to displace itself with respect to the channel component. Alternatively, the central component may be configured to cause separation between a first channel component and a second channel component. The central component may comprise an elongate member having a threaded portion which is configured to be received within a threaded bore of the channel component. The elongate member may be configured such that rotation of the threaded portion causes displacement between the central component and the attachment component in a direction that is parallel to a longitudinal axis of the elongate member. Accordingly, the elongate member may be configured to apply a static force upon the channel component which is configured to engage with the threaded portion. The elongate member may comprise a first and a second threaded portion, arranged at its opposite ends and configured to engage with each of first and second channel components, respectively.

The central component may further comprise an alignment rod extending between the first and second channel components, the alignment rod may be configured to maintain the alignment of the channel components as they move relative to each other, due to the rotation of the threaded portion. The central component may comprise two or more alignment rods. A first and a second alignment rod may be arranged either side of the elongate member to ensure that the components are correctly aligned.

The alignment rod may be cylindrical (i.e. having a circular cross section). It is recognised that the aligning rod may be configured with a cross section that is any one of square, rectangular, elliptical, and hexagonal or any other suitable shape. The alignment rod may be configured with a rounded rectangular cross section including, for example, an obround shaped cross section.

The channel component may be configured (i.e. shaped and/or arranged) to house the hydraulic component. The channel component may comprise one or more holes configured to receive the ends of the alignment rods of the central portion. The hydraulic component may comprise an inflatable structure, such as a balloon. The inflatable structure may comprise an elongate substantially cylindrical balloon including a conical, or frusto-conical, tip at its distal end. A proximal end of the balloon may be connectable to a tube, hose or catheter, which is configured to supply hydraulic fluid to the balloon. When the balloon is in place inside the channel component, an outside edge of the balloon may be aligned with, or extend beyond, an outer wall of the channel component.

The attachment component may be configured to attach the expansion mechanism to a patient's cranial structure. In particular, the attachment may be configured to attach at least one of the channel component and the central component to the cranial structure. The attachment component may comprise a moulded structure which is shaped to match the contours of a patient's palate and/or teeth in order to form a snug fit therewith. The attachment structure may comprise a fastening which is detachable from the moulded structure to enable, for example, a single expansion mechanism to be removable attached to a variety of different moulded structures and fittings. The attachment component may comprise a fastening means configured to attach the expansion mechanism directly to the cranial structure. The fastening means may comprise one or more bone screws. In this way, the attachment component may define at least one of the first and second anchors. The screw may comprise a rounded, or domed, head portion which is configured so as not to offer any sharp edges on which soft tissue may be damaged. A top surface of the domed screw may comprise a hexagonally formed hole, or recess, which may be configured to enable the screw to be turned with a hexagonal tool.

The attachment component may comprise a wing portion, or foot portion, which extends from a main body of the attachment component. The wing portion may comprise a hole configured to receive the fastening means. The hole may comprise a locating slot which is configured to releasably couple the wing portion to a screw. The locating slot may comprise two intersecting circular holes, or bores. A first larger hole may be configured to be larger than a diameter of a head portion of the screw. A second smaller hole may be configured to be larger than a diameter of a shaft portion of the screw and smaller than the diameter of the head portion of the screw. The first hole may be sized to allow it the head portion of the screw to pass through the first hole. The second hole may be configured to receive the shaft of the screw when the attachment component is arranged in its desired portion within a user's mouth. An underside of the domed screw may be configured to be engage with a chamfered edge of the second hole of the locating slot. The wing portion may be substantially aligned with a base of the main body, the wing portion may be configured to extend away along a plane which is substantially parallel to the base of the main body. The wing portion may be integrally formed with a main body of the attachment component. The attachment component may comprise a plurality of wing portions. The wing portions may be arranged at distal corners of the main body of the attachment component. The attachment component may be integrally formed with the channel component.

A cover may be arranged to form a protective shield over at least part of the expansion mechanism. The cover may be arranged to shield at least one of the hydraulic component, the attachment component and the central component. The cover protects the patient's soft tissue from making direct contact with the features of the expansion mechanism. For example, the cover may be arranged to prevent the patient's tongue from contacting any movable component of the expansion mechanism. For example, the hydraulic component, when in use, may be configured to cause periodic displacement of a movable component which could cause harm or discomfort for the patient.

In other, similar embodiments, the device may be used to perform anterior expansion of the maxilla or mandible, i.e. forward growth of the upper or lower jaw, for example to correct an underbite or an overbite. In embodiments in which the device is for anterior mandibular expansion, the device may include a portion having components which are arranged to connect to bone screws which are affixed to an internal surface of the mandible. In some embodiments, the first force and the second force give rise to growth and/or displacement as well as remodelling of the upper/lower surface of the mouth, and of the maxilla/mandible themselves, in a forward-backward direction (anterior-posterior). In these embodiments, the first anchor point may be a tooth or teeth. In such cases the first anchor may be in the form of a wire, band, or other orthodontic fixation configured to wrap around the tooth or teeth, or plates or wires which are configured to abut or engage with the back surfaces of the front teeth. Alternatively, the first anchor may include a moulded plate which is shaped to conform to the back surfaces of the tooth/teeth in order to provide a snug fit, for more efficient force transfer. The second anchor point may be a point on the soft tissue of the user's upper or lower palate, which is located posterior to the first anchor point, and the second anchor may include a screw or equivalent fastener which is configured to contact bone, tooth or soft tissue directly. By contacting the tissue more directly, more efficient transfer of force is enabled.

In the embodiments described above, there are two primary means by which the force generated by the force generator may be transmitted usefully to the first anchor and the second anchor. As outlined above, the force transmitting structure may be in the form of a hydraulic force transmitting structure. Alternatively, a method involving solid components configured to convert one type of motion to another type of motion may be used, i.e. with no hydraulic components. Throughout this application, this will be referred to as a mechanical force transmitting structure. In some embodiments, there may be a hybrid of both a hydraulic and a mechanical force transmitting structure.

As discussed above, it is preferred that the periodic force is maintained in, or converted into a force in the anterior-posterior direction. In order to effect predominantly sideways or lateral maxillary or mandibular expansion, however, the periodic force (and an optional static component) of the force, need to be in a lateral direction. Therefore, the force transmitting structure preferably includes a mechanism for rotating the direction of the force by approximately 90°. Herein, “approximately 90°” should be interpreted also to cover exactly 90°. In addition to this, in some embodiments, the force must be transferred to both the first anchor and the second anchor. The force transmitting structure may include a mechanism for converting the periodic force into the first force which is at approximately 90° to the periodic force, and the second force which is at approximately 90° to the periodic force, and opposite in direction to the first force. The force transmitting structure preferably includes a first force transmitting component arranged to transmit the periodic force from the force generator to the anchor, the first force transmitting component including one or more of a wire, or a piston. Specifically, the force transmitting component is configured to transmit the force in the form of linear reciprocating motion, or periodically oscillating linear tension using a screw thread. Specifically, the mechanism may be configured to convert the linear reciprocating motion into oscillating rotary motion of a rotary component. The rotary component may include a first screw thread. The mechanism may further include a first threaded member having a second screw thread on at least a proximal end, which is complementary to, and engaged with the first screw thread. A distal end of the first threaded member is preferably in contact with the first anchor. The rotary component and the first threaded member are preferably arranged such that rotation of the rotary component is converted to reciprocating linear motion of the first threaded member by the engagement between the first screw thread and the second screw thread. The periodic force is then transmitted to the first anchor by means of this reciprocating linear motion. The rotary component may have a third screw thread, opposite in sense to the first screw thread, and the mechanism may include a second threaded member having a fourth screw thread complementary to, and engaged with the third screw thread. The mechanism may further include a second threaded member having a third screw thread, opposite in sense to the second screw thread. A distal end of the second threaded member is preferably in contact with the second anchor. The rotary component and the second threaded member are preferably arranged such that rotation of the rotary component is converted to reciprocating linear motion of the second threaded member by the engagement between the third screw thread and the fourth screw thread. The periodic force is then transmitted to the second anchor by means of this reciprocating linear motion. Alternatively, the same effect may be achieved with the provision of a first rotary component and a second rotary component. In other embodiments, the mechanism may include one or more worm and pinion gears, or ball and socket connections in order to achieve the same effect.

Other embodiments employ hydraulic transmitting structures including one or more inflatable structures, such as balloons, bellows or other equivalent components. In these embodiments, the force transmitting structure preferably includes a channel or chamber containing a hydraulic fluid, and a piston configured to move the fluid throughout the channel or chamber, thereby causing inflation or collapse of the inflatable structures. In some embodiments, there is a first inflatable structure rigidly connected to the first anchor (e.g. by a first rod, or other rigid body) and a second inflatable structure rigidly connected to the second anchor (e.g. by a second rod, or other rigid body). The inflatable structures here may be in series (i.e. in fluid communication with each other), or may be separate components, removable or permanently attached to separate rigid bodies. Here, rigidly connected should be understood to mean that the two features are connected by an inflexible and/or incompressible connector. The inflatable structures may be located inside grooves or channels. On inflation of the first inflatable structure and the second inflatable structure, the inflating force (i.e. the periodic force, transmitted by e.g. the piston) is transmitted to the first anchor and the second anchor respectively. Alternatively, a single inflatable structure may be rigidly connected to both the first anchor and the second anchor, such that inflation of the single inflatable structure transmits the periodic force to both the first anchor and the second anchor. The, or each, rod may be cylindrical (i.e. having a circular cross section). Alternatively, the rods may each comprise a cross-section which is any one of square, rectangular, elliptical, and hexagonal or any other suitable shape. The, or each, rod may be configured with a rounded rectangular cross section.

Using hydraulic transmitting structures such as those described in the above paragraph, the direction of the periodic force can be more easily altered by an appropriate orientation of the rigid connectors and the inflatable structures. For example, the hydraulic transmitting structure may be used for maxillary/mandibular expansion, anterior expansion or maxillary/mandibular protraction through an appropriate arrangement of inflatable structures and rigid connectors. Throughout the above, the term “rigid” should be understood to mean that a component such as a connector is incompressible over lengths on the scale of the device, ensuring that the force which is applied to one of a component is substantially equal to the force which is transmitted to an opposite end of the component.

In other embodiments, the inflatable structures may be in direct contact with the first anchor and/or second anchor. Alternatively the first anchor and/or second anchor may be formed of the inflatable structures. For example, the device may include a plurality of inflatable structures each configured to abut or engage with a cranial structure such as a tooth, or other feature of the mouth. In some embodiments, when one or both of the first anchor and the second anchor include a moulded structure or plate, one or more inflatable structures may be located on an outer surface of the moulded structure which is configured to face, abut, or engage with either an inner or outer surface of the teeth. It is recognised that one or more inflatable structures may be located on any surface of a moulded structure. It is recognised that the moulded structure may define a structure which is configured to conform to a corresponding surface of the patient's mouth, e.g. a portion of the palate and/or an inner surface of the teeth, and/or a bone interface.

The moulded structure may be configured, when in use, to be secured to the roof of a user's mouth using a fastening means, such as a bone screw, for example. In this way, the fastening means may define at least one of the first and second anchors, as described above. At least one of the moulded structures may be configured to engage with the fastening means in order to secure the moulded structure to the patient's cranial structure. The moulded structure may be configured with a hole, or opening, which is arranged to receive a fastening, e.g. a bone screw. The opening may be arranged at an edge of the moulded structure and configured to be at least partially open at a lateral side. In this way, the opening may enable the moulded structure to be slidably engaged with a fastening which is fixedly attached to the patient's cranial structure. According to an exemplary arrangement, the moulded structure may be shaped to fit the contours of a patient palate, the moulded structure may comprise an opening arranged at a medial end of the moulded structure, the opening being configured to receive a fastening when the moulded structure is moved in a medial direction as it is installed in the patient's mouth.

The moulded structure may comprise one or more moulded plates. According to an exemplary arrangement, the moulded structure may comprise a central moulded plate and a peripheral moulded plate. Each of the moulded plates can be shaped to conform to a corresponding (i.e. selected) surface of the user's mouth, e.g. a portion of the palate and/or an inner surface of the teeth. An inflatable structure, such as a balloon, may be arranged at a joint between the central moulded plate and the peripheral moulded plate. The joint between the central and peripheral moulded plates may define a channel in which the inflatable structure is arranged. The channel may be tapered (i.e. configured such that the width of the channel narrows along the joint between two moulded plates). The tapered channel may narrow from a front portion to a rear portion of the moulded plates. In this way, the tapered channel may be configured to cause greater expansion of the inflatable structure in a wider portion than in a narrower portion. The greater relative expansion of the inflatable structure may cause rotation of the moulded structures relative to each other.

The inflatable structure may be configured to articulate the joint by being inflated and/or deflated with a hydraulic fluid. The inflatable structure may be deflated, or at least only partially inflated, in order to facilitate access to a fastening means configured to secure the moulded structure to the user's mouth. Additionally, or alternatively, the inflatable structure may be inflated to cause the joint to become rigid. Accordingly, the inflation of the inflatable structure causes the peripheral moulded plate to come into intimate contact with the surface of the user's mouth with which it is shaped to conform. In this way, the peripheral moulded plate may be configured to transmit forces generated by the inflatable structure upon the user's mouth. Accordingly, the peripheral moulded plate may define at least one of the first and second anchors, and the inflatable structure may define at least part of the hydraulic force transmitting structure. By inflating the inflatable structure between the central and peripheral moulded plates, the device may be configured to affect maxillary or mandibular expansion of the user's mouth. The channel at the junction between the central and peripheral moulded plates may comprise a substantially straight portion. In use, the straight portion may be arranged at an angle to the midline of the patient's mouth. The junction between the moulded plates may comprise a curved portion, the curvature of which may be configured to at least partially follow the curvature of the lingual plane. Where the device comprises a first and a second peripheral moulded plate arranged either side of a central moulded plate, the channels between the respective plates may be configured such that they are substantially parallel to each other.

In the above described arrangements, it is recognised that the inflatable structures may be configured so as to enable relative movement between the central and peripheral moulded plate structures. Each inflatable structure may be formed of a flexible material which is configured to allow relative displacement of the plates in a lateral and/or vertical direction. The enhanced flexibility provided by the inflatable structures means that, during use, the peripheral moulded plate may be displaced laterally, and also rotated relative to the patient's mouth. In this way, the resulting motion of the peripheral plate is such that it conforms to the shape of the patient's mouth, which thereby reduces the discomfort of the patient.

Furthermore, the above described combination of inflatable and moulded plate structures allows the device to accommodate asymmetric expansion of a patient's cranial structure. For example, such a device is able to accommodate outward rotation of the hemi-maxillae caused by the greater posterior resistance of adjoining structures. In addition, it also allows the device to adapt to differences in relative expansion of the cranial structures (e.g. one hemi maxilla may rotate more than the other) which thereby ensures that an efficient force transfer is maintained.

According to an exemplary embodiment, the first anchor may be defined by a fastening means configured to secure the central moulded plate to the user's mouth and the second anchor may be defined by a portion of the peripheral moulded plate which is shaped to conform to a surface of the user's mouth. In an alternative embodiment, the moulded structure may comprise a further peripheral moulded plate which is arranged at an opposing side of the central moulded plate. According to this arrangement, the first and second peripheral moulded plates may define the first and second anchors, respectively, in that they may both be configured to transmit opposing forces upon opposite sides of the user's mouth. According to an alternative aspect of the invention, the inflatable structure may be arranged at the junction between two structural elements. At least one of the structural elements may define an elongate member which is configured to extend, longitudinally, from the junction. The other of the two structural elements may be a moulded structure which is configured to conform to the shape of a patient's cranial structure. Alternatively, the first and second structural elements may each comprise an elongate member. A free end of the elongate member may be attached to a cranial structure of a patent. In this way, the free end of the elongate member may define at least one of the first and second anchors, as described above. The elongate member may be movably fastened to the other structural element by means of a bolted clasp arrangement, and the inflatable structure may be received through a central aperture of the clasp arrangement.

In a particularly preferred embodiment, the device includes a moulded structure or plate, preferably shaped to conform to the user's mouth. The moulded structure may comprise a recess which is shaped to conform to a user's tooth. The recess may be arranged on a surface of the moulded structure which is arranged to contact at least one of an inner surface, an outer surface, a buccal surface, a labial surface, a lingual surface and an occlusal surface of the tooth. The moulded structure may include a plurality of recesses, each shaped to conform to a plurality of the user's teeth. One or more of the plurality of recesses may preferably include a cantilever structure.

It is recognised a recess may accommodate more than one tooth. The recess may include a cantilever structure which is configured to engage with a user's tooth when the device is in place inside the user's mouth. A surface of the cantilever structure may be shaped to contact a surface of a user's tooth. The cantilever structure may be arranged to contact, face, abut, or engage any surface of the tooth. For example, the cantilever structure may be configured to contact at least one of an inner surface, an outer surface, a buccal surface, a labial surface, a lingual surface and an occlusal surface of the tooth.

In addition to this, the device preferably includes a channel, or groove, and an inflatable structure housed within the channel, the channel being located immediately behind the cantilever structures. The channel may be arranged inside the device, so as to define an internal channel. The channel may be arranged in the moulded structure of the device. The channel is thus preferably curved in order to conform to the curvature of the dental arch. The inflatable structure is preferably arranged such that when it is inflated, it exerts a force on the cantilever structure, the force acting to cause the cantilever structure to bend. Thus, in use, when the inflatable structure is inflated, the force exerted on the cantilever structure is applied to the tooth which rests within the recess, that force acting to give rise to tooth movement. In this way, the cantilever structure may define the first anchor and the tooth may define the first anchor point. The second anchor may be defined by a second cantilever structure. Alternatively, the second anchor may be defined by an inflatable structure which is configured to directly contact a cranial structure, such as a tooth.

In an exemplary arrangement, a first cantilever structure may be configured to contact a first tooth surface and a second cantilever structure may be configured to contact a second tooth surface. The first and second tooth surfaces may each define an inner or an outer surface of the tooth. For example, the first and second cantilever structure may be configured to make contact with two different surface portions of a tooth. The first and second cantilever structures may be configured to apply a separate force to each of the first and second surface portions of the tooth. The second cantilever structure may be configured to apply a force independently from the first cantilever structure. By configuring the first and second cantilever structures to apply force independently from each other, the device may be operable to achieve greater control of the forces which are exerted upon the teeth by the cantilever structures. For example, a first cantilever structure may be configured to apply a first force and a second cantilever force may be configured to apply a second force, wherein the first and second forces comprise different magnitudes and directions. The first and second cantilever maybe configured to apply uneven forces upon a tooth causing translation and/or rotation of the tooth.

A first inflatable structure may be configured to exert a force on the first cantilever structure and a second inflatable structure may be configured to exert force on the second cantilever structure. In this way, inflation of the first and second inflatable structures may be controlled in order to determine the respective forces that are exerted by the first and second cantilever.

The cantilever structure may define an elongate cantilever finger including a first end which is attached to the moulded structure and a second end which is unattached. The unattached end may be moveable relative to the moulded structure to enable the cantilever finger to bend. It is recognised that the cantilever structure may define any structure comprising a fixed end and a movable end (i.e. the movable end being movable relative to the fixed end). The cantilever finger may comprise a fixed end which is wider than it's free end. The cantilever finger may be configured to taper towards the free end.

The recess may be arranged intimately in contact with a surface of the tooth or at some distance from the tooth surface as well as differing in orientation, such as slanted at an angle. Furthermore, the recess comprises of more than one section, in one embodiment a first section may be arranged in a first location of the tooth close to the gumline and a second section may be arranged in a second location substantially away from the gumline. A single cantilever structure may be accommodated within the first and second sections of the recess. The first and second sections of the recess may be interconnected. In some embodiments there may be more than one cantilever finger. Each of these sections may naturally sit at slightly or substantially different angles having been moulded to a tooth or teeth. Alternatively each of the sections could be designed such they sit at a substantially different angle, for example the cantilever finger could alone be substantially orientated and act in upward or downward sloping plane that passes through the local longitudinal axis of the balloon (with respect to its neighbouring sections or the vertical axis.

The area of each of the sections including the cantilever finger could vary for example a thicker cantilever finger will bend less when subject to a force compared to a thinner finger. Reduced flexure of finger may permit less force to be transferred to its paired tooth or teeth, such that varying finger thickness throughout the device would allow for varying force application to teeth when employing a single inflatable structure. The fingers and/or their neighbouring sections can be made out of material of varying stiffness. Furthermore, there may be one, more or no fingers in contact with a tooth or group of teeth. There may be a single or multitude of internal channels, the latter housing a plurality of inflatable structures.

The following features may also vary with respect to the cantilever structures:
Plane of application i.e. angle of the cantilever structures.
Area of applied force i.e. top of tooth, middle of tooth or along gingival edge
Length and number of fingers per tooth
Stiffness of fingers/differing material
Multiple balloons in series or parallel

In addition, the tunnel/groove/channel may be continuous or discontinuous across the midline. The tunnel shape, circumference, length, route may vary. A device may contain a number of tunnels, or channels, with a number of balloons. The channel may be partly or fully partitioned to accommodate one or more balloons.

The internal channel can additionally vary in number of ways for example: route, length, circumference, continuity i.e. does it communicate across a gap between two sections etc.

It is also apparent that certain teeth or teeth can be totally or relatively freed from force application by varying the characteristics of the aforementioned components.

In embodiments, the cantilever structure may be defined as an intermediate structure arranged between the inflatable structure and a cranial structure of the patient. An alternative intermediate structure may comprise a sliding member or a ladle shaped member. In the situation where the intermediate structure comprises a ladle shaped member, the ladle shaped member may comprise a ladle shaped portion which defines the recess of the moulded structure which is shaped to conform to a tooth surface.

The intermediate structure may be defined by a weakened portion of the tooth facing surface, or wall, of the recess of the moulded structure. In this way, the tooth facing wall of the recess may comprise a region which is scored or punctured in order to reduce its rigidity relative to the surrounding recess wall. The weakened surface portion may be defined by two or more cantilever fingers which extend across an aperture in the recess wall. Each of the cantilever fingers may be connected to another cantilever finger in order to form a flexible lattice arrangement. The lattice of cantilever fingers may define a cross-shaped arrangement.

Each of these alternative intermediate structures may be configured to receive force from the inflatable structure and to transmit that force to a tooth, for example. In use, as the inflatable structure is inflated, and so expands outwards, its outer surface may exert pressure on an inner surface of the intermediate structure, causing an outer surface of the intermediate structure to press against the surface of a tooth, thus causing tooth displacement and maxillary expansion.

It is recognised that the intermediate structure may further define any recess, or tooth receiving portion, of the moulded structure which is configured, when in use, to be arranged between the inflatable structure and a surface of a user's tooth, and which may be configured to transmit force therebetween. Such intermediate structures may be necessarily configured to contact any surface of the tooth which may be manipulated in order to enact tooth displacement. For example, an intermediate structure may be configured to contact at least one of an inner, an outer and an occlusal surface of a user's tooth.

In the embodiments described above, it is preferable that the first anchor, the second anchor, at least a portion of the force transmitting structure (preferably the mechanism for converting the periodic force into the first force which is at approximately 90° to the periodic force, and the second force which is at approximately 90° to the periodic force, and opposite in direction to the first force or the inflatable structures) are sized to fit inside a user's mouth. Such arrangements may be referred to as intraoral appliances, since the bulk of the device is located in a user's mouth during use. This is advantageous because it allows for a more compact device. It should also be accepted that intra-oral covers any combination of balloon arrangements such as opposing balloons either side of a tooth, contacting a group of teeth, a balloon whereby its expansion causes retraction of a tooth and balloons located over the gums of the maxilla for example. The same principle applies to the cranium. In embodiments, in which the device includes a hydraulic force transmitting structure, the change in direction of the force may be dictated by the shape of the inflatable structure used.

In some embodiments, the device may include a moulded plate which is shaped to conform to the surface of the user's teeth. In some embodiments, it is preferable that the moulded plate conforms to the surface of the user's teeth in a flush manner, in order to ensure maximum transmission of force.

The moulded plate may be shaped to conform to the inner surface of the teeth, the upper most portions of the teeth and part of the outer surface of the teeth. In this way the tooth is partially encapsulated. When force is applied to the inner surface of the tipped tooth the outer section of the moulded plate acts as a ‘rotational stop’ limiting further tipping and eventual uprighting of the tooth. The device can effect translation when used on teeth which are already in the correct plane i.e. not tipped. By minimally increasing the distance between the external wall of the moulded plate and outer surface of the tooth the device can accommodate both tooth tipping and uprighting i.e. tooth movement and tipping can occur till the tooth comes into the contact with wall after which uprighting occurs. At this point should further expansion be desired another device can be moulded. It is also appreciated that one or more of the recesses may possess these features to a lesser, greater extent or be totally absent e.g. greater wall height neighbouring outer surface of tooth or absent cantilever fingers. The reverse arrangement can be also be used to ‘push’ teeth ‘inwards’.

However, in some embodiments, an additional effect of either preventing or remedying tooth tilt may be achieved if the moulded plate is shaped to conform to the tooth only at an upper portion of the tooth, with a spacing distance between the inner surface of the moulded plate and a surface of the tooth increasing with distance from the upper portion of the tooth. Alternatively, the moulded plate may be shaped to conform to the tooth only at a lower portion of the tooth, with a spacing distance between the inner surface of the moulded plate and a surface of the tooth increasing with distance from the lower portion of the tooth. In this way, as the device is used to e.g. effect maxillary expansion, an outward force is only applied to, say, the lower portion of the tooth after some movement of the upper portion of the tooth, e.g. to correct tilt of that tooth.

Put differently, the moulded plate may be shaped to conform to the inner surface of the teeth, the upper most portions (tips) and part of outer surface. In this way the tooth is partially encapsulated. When force is applied to the inner surface of the tipped tooth the outer section of the moulded plate acts as a rotational stop limiting further tipping and eventual uprighting of the tooth.

The device can effect translation when used on teeth which are already in the correct plane i.e. not tipped by limiting or preventing any rotational movement.

At this point should further expansion be desired another device can be moulded. It is also appreciated that one or more of the recesses may possess these features to a lesser, greater extent or be totally absent e.g. greater wall height neighbouring outer surface of tooth or absent cantilever fingers. The reverse arrangement can be also be used to push teeth medially or inwards, i.e. towards the centre of the mouth. Explanatory drawings relating to the geometry of the cantilever structures are set out in FIG. 37.

In the embodiments described above, the cranial structure which is being restructured is located between the first anchor point and the second anchor point. However, the core principle of the invention applies equally well to cases in which the cranial structure to be altered does not lie between the two anchor points. Accordingly, a second aspect of the present invention provides a device for cranial restructuring, by the expansion, compression, or flexure of a cranial structure having a first anchor point, the device including: a head support, having a head-receiving portion which is configured to receive a portion of a user's head; a force generator configured to generate a periodic force; a first anchor for attachment to the first anchor point; a force transmitting structure, connected to the force generator, and configured to transmit the periodic force to the first anchor in a restructuring direction; wherein the head support includes: a restriction means configured, in use, to prevent or restrict movement of the user's head in the restructuring direction, when the periodic force is being applied. In a very simple arrangement, the second aspect of the invention may be realized by an arrangement in which the force generator(s) are connected, via the force transmitting structure to one or more bone screws which are in place in e.g. lateral walls of the maxilla, via one or more wires, and an oscillating force is applied. In such cases, as discussed in more detail below, the restriction means may be in the form of a friction-providing device, or just the weight of a user's head which prevents oscillation of the head as a result of its inertial mass. Securing straps may also be utilised to stabilise the head.

Where compatible, the optional features set out above with reference to the first aspect of the invention may apply equally well to the second aspect of the invention, and for conciseness will not be repeated here. In these embodiments, the head support includes a restriction means which prevents the periodic force from causing only displacement of the user's whole head or the device. In other words, the restriction means receives at least a component of the reaction to the periodic force. Examples of restriction means will be discussed in more detail later. Devices of the second aspect of the invention are particularly useful for maxillary protraction.

The device may take the form of a cradle device including a head support and a rail. The head support is preferably configured to be tightened around a user's head, preferably in a manner where force is exerted against the side surfaces of the user's head and face. This means that friction between the user's head and the inner surfaces of the head support acts to prevent anterior-posterior motion of the user's head when an anterior-posterior force is applied. The rigidity of the structure and its securing mechanism also help resist sideways motion when a lateral force is applied. In other words, in such embodiments, the inner surfaces of the sides of the head support from at least part of the restriction means. More generally, it may be said that the restriction means is configured to restrict or prevent movement of the user's head by friction. In order to be effective, the frictional force generated by the contact between the user's head and the inner surfaces of the head support should be greater than the periodic force applied to the first anchor point.

Alternatively, the restriction means may include an abutment surface configured to abut the user's head in use, wherein contact with the abutment surface is configured to prevent or restrict movement of the user's head in the restructuring direction. In some embodiments, the weight of the user's head alone may provide the restriction means.

The rail is preferably connected to the head support by one or more connectors. In such embodiments, the force generator may be in the form of a motor which is connected to both the head support and a proximal end of the connector. Alternatively, the force generator may be in the form of a hydraulic pump, also connected to a proximal end of the connector. In some embodiments, there may be a first force generator and a second force generator, connected to the rail respectively by a first connector and a second connector. The rail is preferably attached to the distal end of the connector or connectors. The rail and the first and/or second connector may form part of the force transmitting structure, which may further include one or more additional connectors, e.g. a third connector, a proximal end of which is preferably attached to the rail, and a distal end of which includes the first anchor, which as discussed earlier in this application, is configured for attachment to a first anchor point. The first anchor point may be in the form of a tooth, the maxilla, the mandible, part of the occlusal plane, a zygoma, an upper portion of the skull, the nose, or other structure.

For symmetrical protraction, the device preferably includes a second anchor for connection to a second anchor point of the cranial structure. The device may further include a fourth connector which includes the second anchor at its distal end. The second anchor point may be any of the same cranial structures listed in the previous paragraph for the first anchor point.

In some embodiments, the device may include a plurality of rails, so that protraction of more than one cranial structure can be achieved at the same time. There is preferably at least one, and preferably a pair, of force generators associated with each of the plurality of rails, connected to the rails via connectors.

In some embodiments, it may be possible to vary the height of the rail. Here, by “height”, we are referring to the extent in the superior-inferior direction. In this way, one device may be used to effect restructuring of different cranial structures without having to use an entirely different device. In order to achieve this, the connector or connectors may be rotatably attached to the head support so that the assembly comprising the connector or connectors and the rail can be rotated to the desired position for cranial restructuring. Alternatively, the assembly including at least the connectors and rail, and preferably the force generator or generators too, may be translated in superior-inferior direction, e.g. on a dedicated structure. In some embodiments, the rail may be mounted on an assembly on which it may be rotated and translated, to allow even greater flexibility of movement.

In some embodiments, the restriction means may be located on a rail. Preferably, the restriction means is movable along the rail, so that it may be located in the optimum position for the particular cranial restructuring which is taking place. There may be a plurality of restriction means, e.g. to ensure symmetrical operation.

The present invention also covers devices which may be used for cranial compression. While embodiments of some aspects of the invention set out above may be used to perform compression, the majority of them are directed towards systems which exert tensile forces on cranial structures. A third aspect of the invention provides a device for cranial compression including: a head support unit configured to exert a compressive force on at least a portion of a user's cranium; a force generator configured to generate a periodic force; a force transmitting structure configured to transmit the periodic force to the user's cranium.

In some embodiments of the third aspect of the invention, the head support includes or is in the form of a helmet, the inner surface of which is shaped or moulded to conform to the outer surface of a user's head. By moulding the helmet to conform closely to the shape of the user's head, it is possible to ensure a tight fit, which in use will exert compression roughly equally in all directions on a user's cranium. Regions may be omitted to limit force application to specific areas of the head. Embodiments of the third aspect of the invention are likely to be used to correct a particular cranial deformity, which will require a compressive force to be applied in a particular direction on a specific part of the user's cranium. Benefit may also be obtained by action on soft tissue structures. It is therefore highly preferably that the head support includes a first portion which is positioned to cover the cranial structure in question, and a second portion which is positioned opposite or substantially opposite the cranial structure in question. Here, “opposite” should be understood to mean that the first portion and second portion are located on opposite sides of the user's cranium, in use. This ensures that when a compressive force is applied, by the first portion, to the cranial structure in question, the second portion is able to provide the required reaction force in order to maximize the effect of the compressive force.

In some embodiments, rather than the head support being in the form of a moulded helmet, the head support may include a first section and a second section, which are movable relative to each other, and means for connecting the first section to the section in a manner wherein the inner surfaces of the first section and the second section are configured to apply a compressive force on the user's cranium. In some embodiments, the first section and the second section may be joined to each other at a hinge. Then, the first section and/or the second section may include locking means for securing the first section and the second section in a region opposite from the hinge. For example, the first section may be shaped to receive the back of a user's head, so that the user can lie face-up with their head in the first section. The hinge may be located in a region corresponding to the top of the user's head, so that once the user has put their head in place in the first section, the second section can be lowered (i.e. pivoted about the hinge) over their head, and connected to the second section via suitable locking means.

In other embodiments, the head support may further include a third section, the second and third sections being connected to the first section via hinges. Like the embodiment described in the previous section, the first section may be shaped to receive the back of a user's head, so that the user can lie face-up with their head in the first section. The hinges may be located in a region corresponding to the left and the right of the user's head, so that once the user has put their head in place in the first section, the second section and third section can be raised around the sides of the user's head, and secured to each other via suitable locking means. This may be used for e.g. lateral compression of the user's cranium. These embodiments may also readily employ hydraulic elements to actuate other components as well directly apply force to anchor points e.g. by including inflatable structures on the surface of structures contacting the skull

As with the force transmitting structures of the previous aspects of the invention, the force transmitting structure of the third aspect of the invention is preferably a hydraulic force transmitting structure. It preferably includes one or more inflatable structures such as balloons or bellows, which are positioned on the inner surface of the head support in a location corresponding to the cranial structure in question. The force generator is preferably configured to periodically inflate and deflate the inflatable structure or structures in order to superpose a periodic force onto the static compressive force applied by the head support.

The previous three aspects of the invention have focused on the periodic nature of the force. However, it has been noted by the present inventors that advantageous effects may be provided by a device capable of imparting any force (e.g. a constant force, a continuous force, a decaying force, or a periodic force as defined earlier in this application) using a hydraulic force transmitting structure. Using a hydraulic force transmitting structure leads to more efficient and more easily controllable transfer of the force from a force generator to a cranial structure. The following aspects of the invention are focused on devices which impart a general force, be it constant or otherwise, using a hydraulic force transmitting structure. It is also appreciated that all aspects including the electromechanical variants can be used to generate a constant force as well as continuous rate of displacement i.e. a constant rate.

Accordingly, a fourth aspect of the invention provides a device for cranial restructuring, by the expansion, compression, or flexure of a cranial structure located between a first anchor point and a second anchor point, the device including:
a force generator configured to generate a force;
a first anchor for attachment to the first anchor point;
a second anchor for attachment to a second anchor point;
a hydraulic force transmitting structure, connected to the force generator, and

configured to transmit the periodic force to the first anchor and the second anchor thereby putting the cranial structure in at least one of: tension, compression or flexure.

A fifth aspect of the invention provides a device for cranial restructuring, by the expansion, compression, or flexure of a cranial structure having a first anchor point, the device including: a head support, having a head-receiving portion which is configured to receive a portion of a user's head; a force generator configured to generate a force; a first anchor for attachment to the first anchor point; a hydraulic force transmitting structure, connected to the force generator, and configured to transmit the periodic force to the first anchor in a restructuring direction; wherein the head support includes: a restriction means configured, in use, to prevent or restrict movement of the user's head in the restructuring direction, when the periodic force is being applied.

A sixth aspect of the invention provides a device for cranial compression including: a head support unit configured to exert a compressive force on at least a portion of a user's cranium; a force generator configured to generate a force; and a hydraulic force transmitting structure configured to transmit the periodic force to the user's cranium.

The skilled person will appreciate that the optional features set out earlier in this section in respect of the first, second and third aspects of the invention may also apply to the fourth, fifth and sixth aspects of the invention. The skilled person will note in particularly that the optional features of the first aspect of the invention are particularly (but not exclusively) applicable to the fourth aspect of the invention, the optional features of the second aspect of the invention are particularly (but not exclusively) applicable to the fifth aspect of the invention, and the optional features of the third aspect of the invention are particularly (but exclusively) applicable to the sixth aspect of the invention.

Devices according to the present invention may be made entirely of MRI-safe materials, such as polymers and/or non-magnetic materials. In this way the device could be utilized by a user who is simultaneously undergoing MRI scanning.

It should be noted that in some cases, more than one device according to any embodiment of any aspect of the invention may be used in conjunction with each other in order to provide cranial restructuring. For example, subjecting the maxilla to tension using an embodiment of any of the first and fourth aspects of the invention could take place during the same routine as cranial compression. Devices of the present invention are preferably programmable to work synchronously and/or simultaneously.

### BRIEF DESCRIPTION OF THE DRAWINGS

The present invention will now be described with reference to the accompanying drawings, in which:

FIGS. 1A and 1B show an example of a device for maxillary expansion.

FIGS. 2A and 2B shown an example of an alternative device for maxillary expansion.

FIG. 3 shows an example of another alternative device for maxillary expansion.

FIG. 4 shows an example of another alternative device for maxillary expansion.

FIG. 5 shows an example of a similar device, which is used for mandibular expansion.

FIGS. 6A and 6B show examples of an alternative device for maxillary expansion.

FIGS. 7A, 7B and 7B show examples of an alternative device for maxillary expansion.

FIGS. 8A and 8B show an alternative device for maxillary expansion.

FIG. 8C shows several options for various components of a device for maxillary expansion.

FIG. 9 shows an example of a device for anterior maxillary expansion.

FIG. 10 shows an example of a device for anterior maxillary expansion.

FIG. 11 shows an example of a device for maxillary expansion including two moulded plates and a plurality of balloons.

FIGS. 12A and 12B shows an alternative device for maxillary expansion, using a plurality of balloons.

FIGS. 12C and 12D shows an alternative device for maxillary expansion, including recesses containing cantilevered fingers and an elongate balloon.

FIGS. 12E to 12H show four further examples of devices for restructuring of the occlusal plane.

FIG. 12I shows a close-up of a depression which may be found in the devices of e.g. FIGS. 12F to 12H.

FIGS. 12J and 12K each show a cross section of an alternative device for maxillary expansion, including an inflatable structure and a sliding member.

FIG. 12L shows a cross section of an alternative device for maxillary expansion, including an inflatable structure and a ladle shaped member.

FIG. 13A shows an example of a device which may be used to effect maxillary expansion.

FIG. 13B shows a balloon which may be used with the device of FIG. 13A.

FIG. 14A shows a device which may be used to separate bones of the cranium.

FIG. 14B shows a close-up view of an expansion mechanism which may be used in various embodiments of the present invention.

FIGS. 15A and 15B show views of an alternative expansion mechanism which can be used in various embodiments of the present invention.

FIGS. 15C to 15E show a slotted screw hole arrangement.

FIGS. 16A to 16D show examples of a device which may be used for maxillary expansion, minimizing or reversing the effect of the tooth tilting.

FIGS. 17A to 17C show various means of mounting devices according to the present invention to a user's body.

FIG. 18 shows an example of a device which may be used for maxillary protraction.

FIG. 19 shows an example of a crossbar structure which may be used in the device of FIG. 18.

FIGS. 20A and 20B show alternative crossbar structures which may be used in the device of FIG. 18.

FIG. 21 shows an example of an alternative device which may be used for maxillary protraction.

FIG. 22A shows an example of a hydraulic crossbar structure which may be used in device such as that shown in FIG. 21.

FIGS. 22B and 22C show close-up views of components in the crossbar structure of FIG. 22A.

FIG. 23 shows an alternative device which may be used for maxillary protraction in which there are two levels of attachment points on the cranium.

FIG. 24 shows an alternative device which may be used for maxillary protraction.

FIGS. 25A to 25C show schematic views of devices in which the force is transferred to the zygoma.

FIGS. 25D to 25F are schematic diagrams illustrating the placement of the device between the zygoma and maxilla.

FIG. 26 shows an alternative device which may be used for maxillary protraction.

FIGS. 27A and 27B show adjustable devices in which the attachment point is the nasal bone.

FIG. 28 shows an adjustable device in which the attachment point is an intra-nasal structure.

FIGS. 29 to 32B show arrangement including a plurality of rails which may be used for various types of cranial restructuring.

FIGS. 33A and 33B show helmet devices which may be used for cranial compression.

FIGS. 34A to 34E show examples of arrangements in which a user may place their head to receive compression or other types of cranial restructuring from an external device.

FIG. 35 shows an alternative device which may be used for cranial compression in the superior-inferior direction.

FIGS. 36A and 36B are schematic diagrams illustrating how the device of FIG. 35 may be used.

FIG. 37 shows a plurality of different geometries of cantilever fingers in recesses.

FIGS. 38A and 38B show a Matthew-Tessiers distractor which may be used in combination with embodiments of the present invention.

### DETAILED DESCRIPTION OF THE DRAWINGS

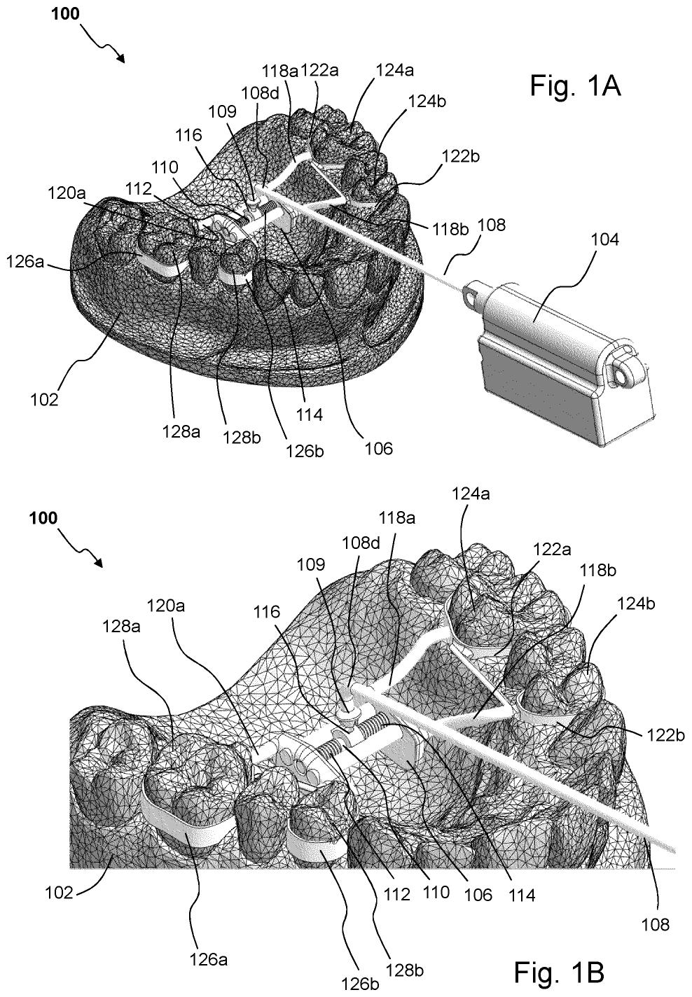

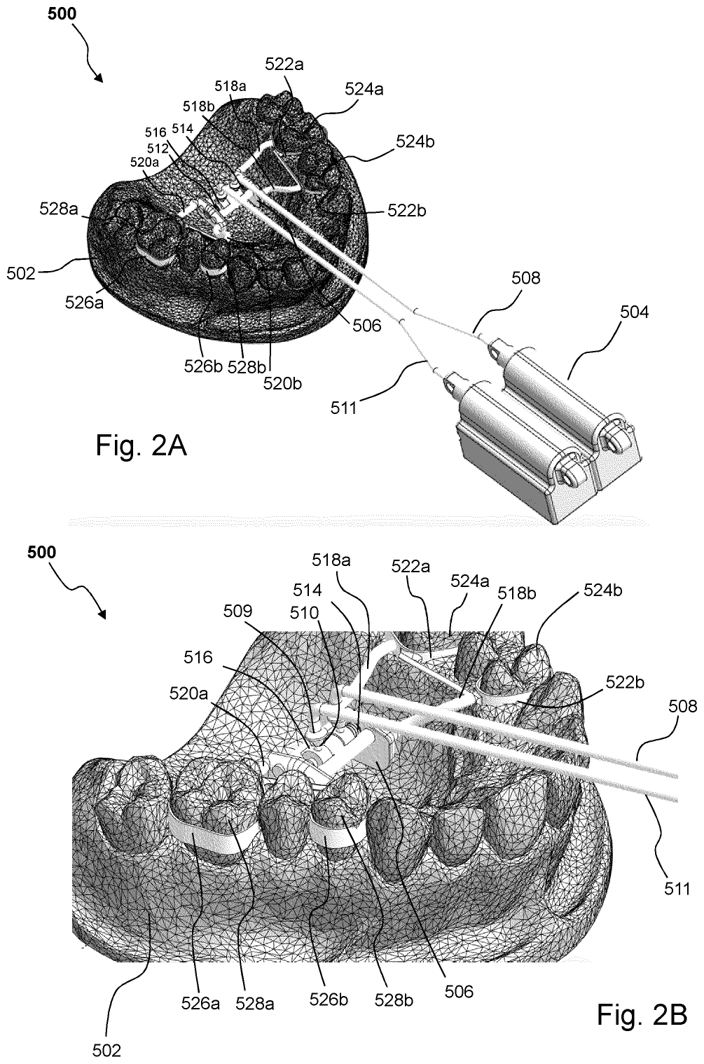

FIGS. 1A and 1B show an embodiment 100 of a device of the present invention which may be used to widen the maxilla 102 (shown inverted in these drawings), but which may be used equally well to widen the mandible. The device includes an extraoral force generator 104 which is connected to intraoral portion 106 via connecting rod 108. Force generator 104 may include a motor (not shown). The intraoral portion 106 includes a central portion 110 having two screw threads 112, 114, and a central ball and socket 116. Distal end 108d of the connecting rod 108 includes a screwdriver portion 109 configured to rotate central ball and socket 116. The intraoral portion 106 also includes two pairs of laterally extending arms 118a, 118b, and 120a (and a further arm which is not visible in the drawing). Arms 118a, 118b are connected respectively to first and second loops 122a, 122b which themselves are looped securely around teeth 124a, 124b. Similarly, arms 120a and the second arm which is not visible are connected respectively to third and fourth loops, 126a, 126b which are looped securely around teeth 128a, 128b. In operation, oscillatory rotation of a motor within extraoral force generator 104 causes rotation of the distal end 108d of connecting rod 108, such that the screwdriver portion 109 causes the central ball and socket 116 to rotate in an oscillatory manner. This oscillating motion in turn gives rise to lateral forces on screw threads 112, 114 which is transmitted into the laterally extending arms 118a, 118b, 120a and the second arm which is not visible, and loops 122a, 122b, 126a, 126b to apply a periodic laterally-outward force on teeth 124a, 124b, 128a, 128b. This laterally outward force can act to displace the teeth and/or widen the maxilla. The device 500 shown in FIGS. 2A and 2B is substantially the same as the device 100 shown in FIGS. 1A and 1B, except there are two rods 508, 511, allowing for independent force transmission to the teeth 524a, 524b and 528a, 528b. Similar reference numerals as those used in FIGS. 1A and 1B indicate similar features in FIGS. 2A and 2B. A useful effect may also be obtained as a result of continuous rotation, rather than oscillatory rotation. For example, the device may rotate 90° over 12 hours. The rate of rotation may be variable. The device may also be used to give rise to an inward force, with slight rearrangement of the central mechanism (e.g. to reverse direction of the screw threads 112, 114 or reverse the direction of actuation). Such a rearrangement is well within the remit of the skilled person.

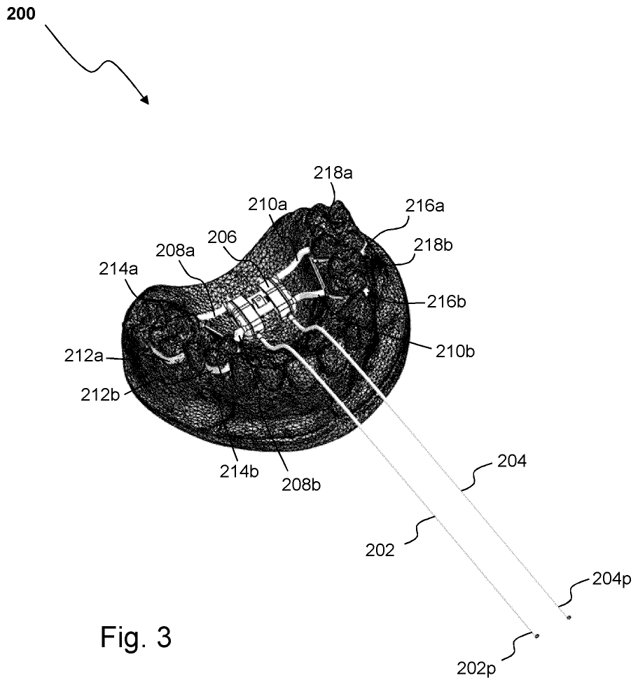

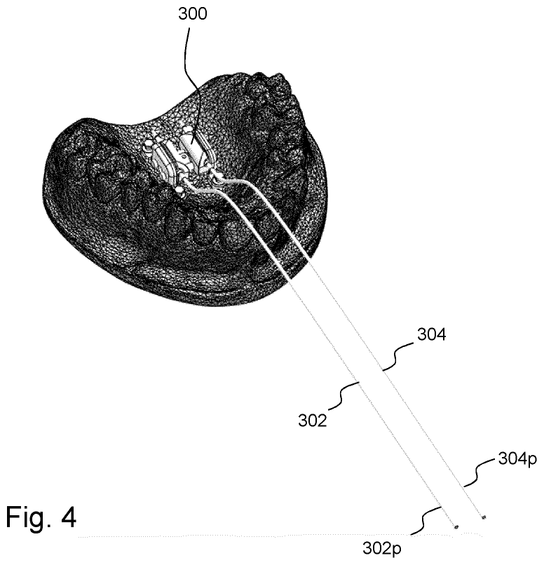

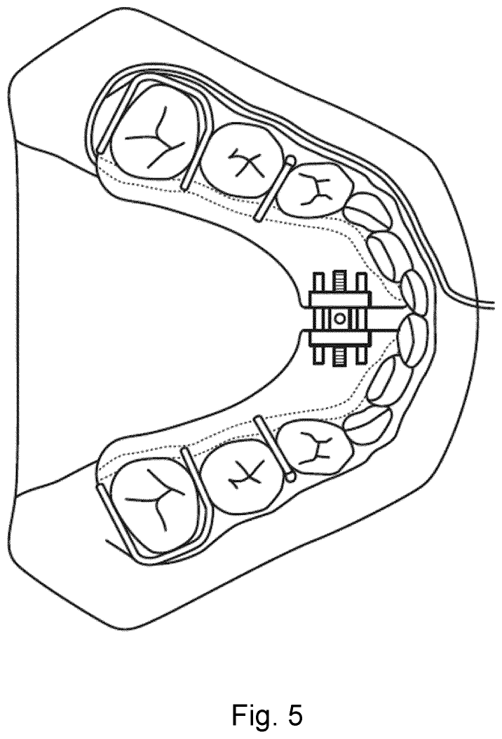

FIG. 3 shows an alternative embodiment 200, in which the period force is transmitted hydraulically. The device 200 includes two tubes 202, 204, each connected to intraoral portion 206. As in FIGS. 1A and 1B, the intraoral portion 206 includes two pairs of laterally extending arms 208a, 208b, 210a, 210b. Arms 208a, 208b are connected respectively to first and second loops 212a, 212b which themselves are looped securely around teeth 214a, 214b. Similarly, arms 210a, 210b are connected respectively to third and fourth loops, 216a, 216b which are looped securely around teeth 218a, 218b. Though not shown in FIG. 3 the tubes 202, 204 are connected at their proximal ends 202p, 204p to a force generator, which may include a pump. Specifically, the tubes 202, 204 may contain a hydraulic fluid, such that the action of the pump causes movement of the fluid back and forth within the tubes 202, 204. In some embodiments, the pump may be a manual pump with an indicator (either visual, audio, tactile or combination thereof) informing a user when to pump. An adjustable pressure regulator may also be used to regulate pressure generated through irregular user activity. However, in preferred embodiments, the pump is an automatic, preferably electrical or electromechanical pump. The movement of the fluid within tubes 202, 204 is converted into laterally outward forces on the laterally extending arms 208a, 208b, 210a, 210b, and loops 212a, 212b, 216a, 216b to apply a periodic laterally-outward force on teeth 214a, 214b, 218a, 218b. This laterally outward force can act to displace the teeth or widen the maxilla. FIG. 4 shows substantially the same device 300 as the device 200 in FIG. 2, except in this case, the device 300 is anchored not to the teeth, but is mounted to the bone at the roof of the mouth, via the soft tissue covering the top of the mouth. In alternate embodiments, the device could also be anchored to the walls of the mouth and/or the teeth.

FIG. 5 shows a device similar to those shown in FIGS. 1A and 1B, except it is shown here in place on a mandible instead of a maxilla. The internal channel may cross the midline for example in the form of a flexible connection or each section may have its own internal channel. The skilled person will appreciate that the description of FIGS. 1A and 1B applies equally well here. Furthermore, it will be appreciated that the devices of FIGS. 2A to 4 could also be modified straightforwardly for use with a mandible, rather than a maxilla.

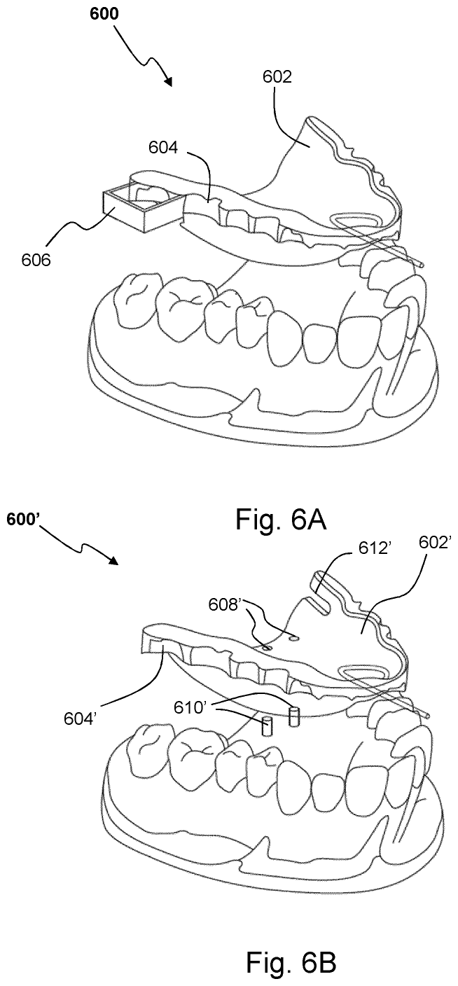

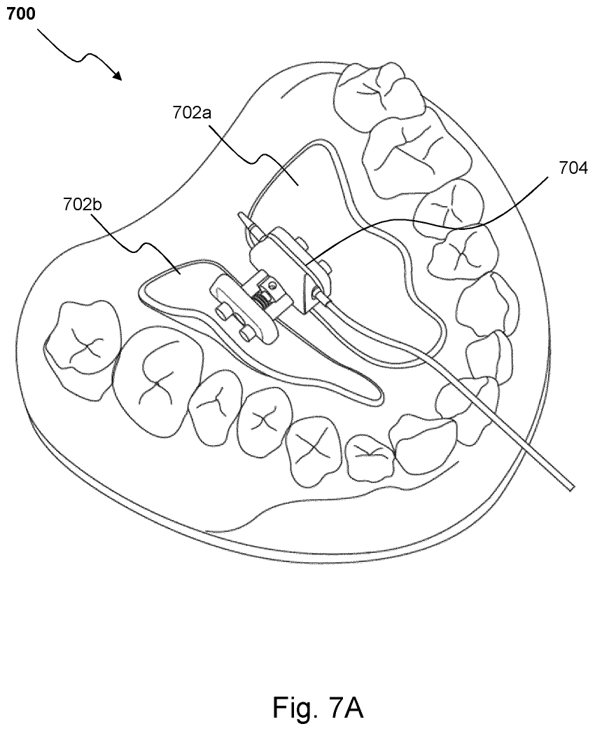

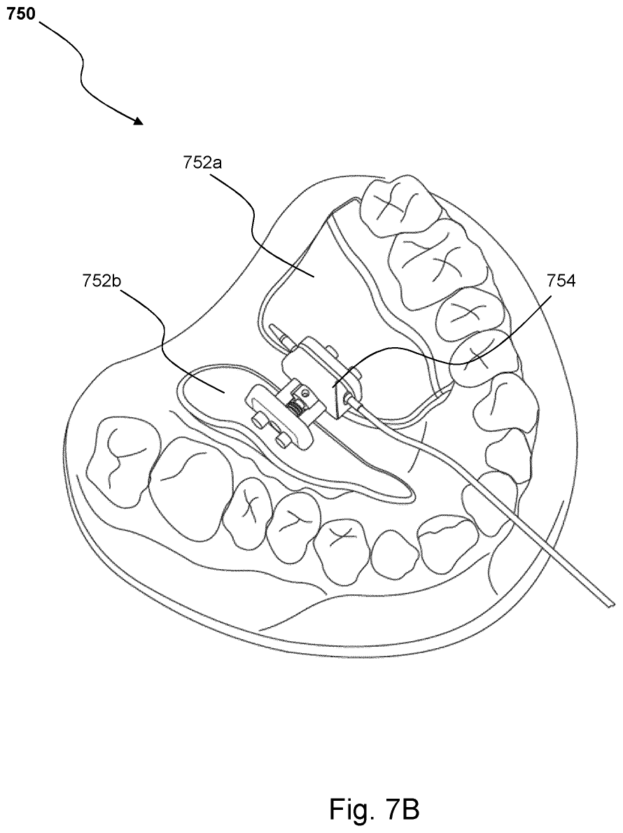

FIG. 6A shows an intraoral device 600 for use in expanding the dental arch of a user (shown inverted). The device 600 includes a plate 602 which is shaped to conform to a user's palate. The moulded plate 602 includes a plurality of recesses 604 around its periphery, each of the recesses 604 shaped to conform to the surface of a respective tooth. In the device 600 of FIG. 6A, an optional structure 606 is also present which is shaped to wrap around the outer surface of a tooth. The parts of the device which are arranged to contact the teeth of the user each include a balloon, via which the force may be transmitted to the teeth through an aperture in the recess between the balloon and the tooth surface. Specifically, the force transmitter in the present case is hydraulic. In FIG. 6A, an additional section is provided for anchoring to the tooth of a user. The device 600′ of FIG. 6B is similar to the device 600 of FIG. 6A, with similar reference numerals depicting similar features. In FIG. 6B, the device 600′ further includes two holes 608′, which are located to receive screws 610′ which are fixed to the user's palate. The device 600′ also includes a slot 612′ which is shaped to receive a screw (not shown) in the user's palatal wall. The skilled person will appreciate that the embodiments of FIGS. 6A and 6B may be combined, and that all of the features shown are optional.

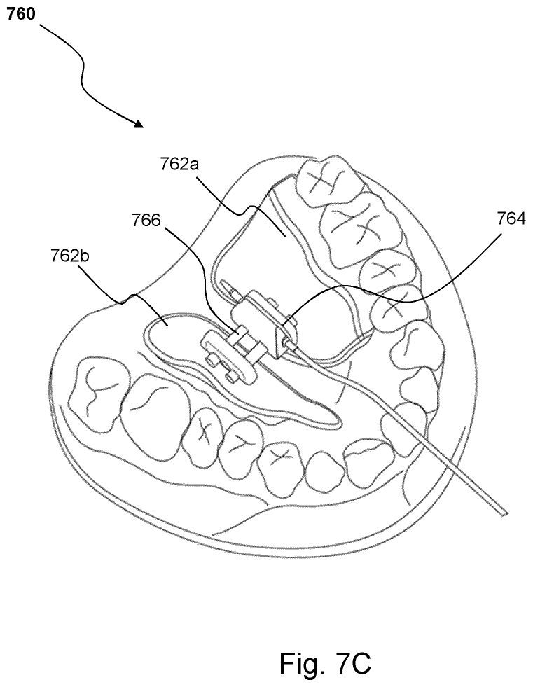

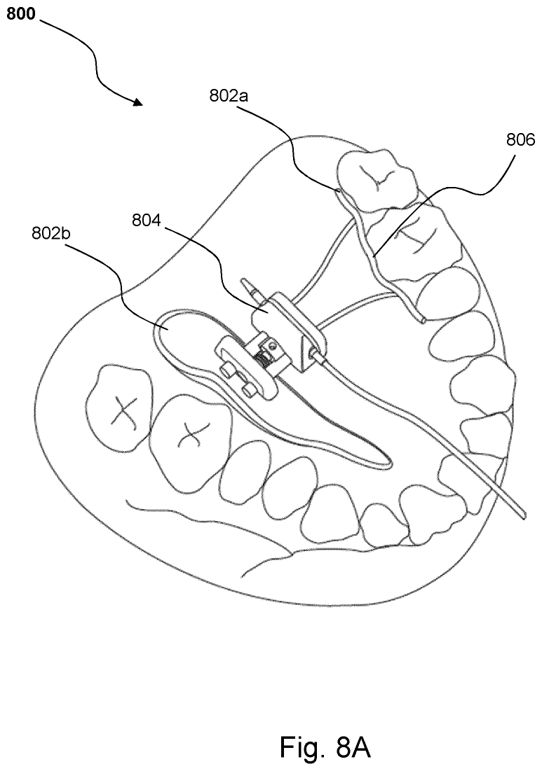

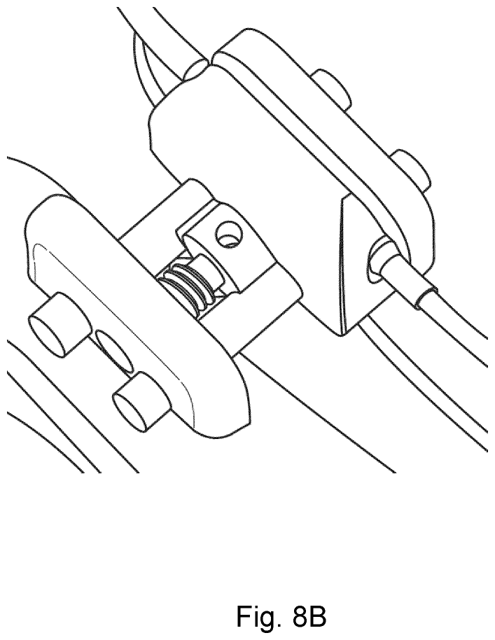

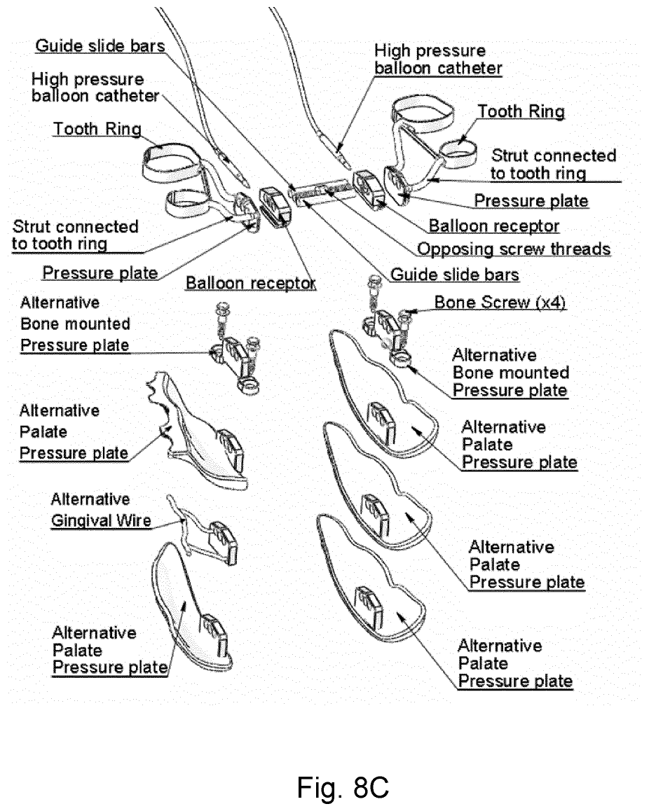

FIGS. 7A, 7B, 7C and 8A show additional examples of devices 700, 750, 760, 800 each of which includes a moulded plate 702a and b, 752a and b, 762a and b, 802a and b. The expansion mechanism 704, 754, 764, 804 of each device is as shown and described in detail with reference to FIG. 8B (see below). FIGS. 7A, 7B, 7C and 8A differ in the areas to which the force is applied within the mouth:
In the device 700 of FIG. 7A, the force is applied via two plates 702a, 702b, which are moulded to conform to the inner surface of the palate. This ensures an even distribution of force across the whole palate, which ensures an even expansion.
In the device 750 of FIG. 7B, there are two moulded plates 752a, 752b. The device 750 of FIG. 7B differs from the device 700 of FIG. 7A in that one of the moulded plates 702a is replaced by a moulded plate 752a which, in addition to conforming to the inner surface of the palate, is also shaped to conform to the inner edges of the teeth. This means that the force is applied to both the inner surface of the palate, and the teeth. In some embodiments, the moulded plate may also include a section which is shaped to conform to the inner surface of the teeth proximal to the gum line. Such a moulded plate is not specific to the embodiments shown in FIG. 7B, but could be present in any compatible embodiment. In the embodiment shown, the moulded plate is in contact with four teeth, but the skilled person understands that embodiments of the invention need not be restricted to contacting four teeth; other numbers of teeth, e.g. one, two, three, five or six are envisaged.
In the device 760 of FIG. 7C, there are two moulded plates 762a, 762b. The device 760 of FIG. 7C differs from the device 700 of FIG. 7A in that the expansion mechanism 764 does not comprise a screw thread. The expansion mechanism comprises a pair of elongate aligning rods 766 which extend between two hydraulic force actuating components.
In the device 800 of FIG. 8A, there is only a single moulded plate 802b. The other moulded plate is replaced entirely by a wire structure 806, where wire 802a is shaped to conform to the inner surfaces of the teeth, and does not contact directly the inner surface of the palate. In the embodiment shown, the wire is in contact with three teeth, but the skilled person understands that embodiments of the invention need not be restricted to contacting three teeth; other numbers of teeth, e.g. one, two, four, five or six are envisaged.

FIG. 8B shows a close up view of the central mechanism which can be employed in the devices shown in FIGS. 7A to 8A. In this embodiment, the screw provides the static “background” force, which can be modified externally by tightening or loosening using the hole in the upper surface of the head of the screw. In use, the hydraulic component provides an additional periodic force, which is effectively superposed onto the static force to generate a periodic non-zero force. In the embodiments shown, one side of the palate represents the first anchor point, and the part of the mouth against which the other component rests represents the second anchor point, and the force generated acts to put the palate which is located between those areas into tension. To a certain extent, the dental arch is also put in flexure which also acts to widen the palate. Various examples demonstrating the modularity of the invention are shown in FIG. 8C.

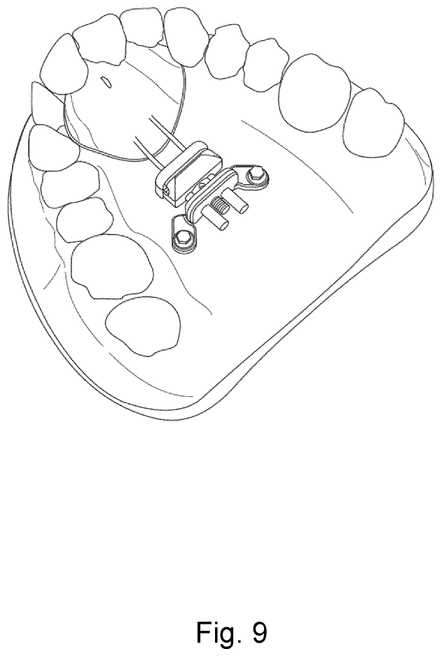

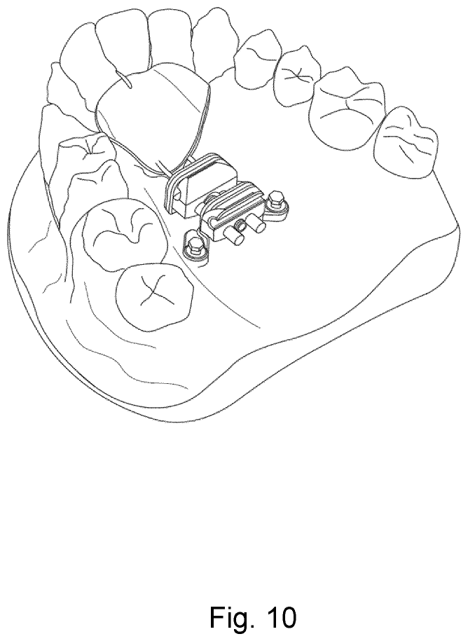

The embodiments shown in FIGS. 9 and 10 are somewhat similar to those shown in FIGS. 7A to 8A, except the devices are oriented in an anterior-posterior direction on the palate, in order to effect expansion of the anterior maxilla. In the configurations shown in FIGS. 9 and 10, the central mechanism is affixed to the upper surface of the mouth (the maxilla is shown inverted) via e.g. bone screws, though of course other fasteners are equally suitable. The mechanism may be the same or substantially the same as the mechanism shown in FIG. 8B, with a background static force being produced by a screw, and the additional periodic component being applied hydraulically. In both FIG. 9 and FIG. 10, the portions of the palate to which the bone screws are attached form e.g. the first anchor point, and the bone screws the first anchor(s). The second anchor differs between FIG. 9 and FIG. 10, though in both cases, moulded plates are used:
In FIG. 9, the second anchor is in the form of a moulded plate, the anterior edge of which is shaped to conform to the rear sides of the front teeth. A portion of the moulded plate is also shaped to conform to the surface of the palate too. This ensures an even transfer of force to the teeth and the palate, for even maxillary expansion.
In FIG. 10, the moulded plate does not contact the teeth, and instead contacts only the soft tissue of the upper palate where bone screws may be situated (not shown).

In the embodiments shown in FIGS. 9 and 10, the skilled person is well aware that the lateral extent of the moulded plates may vary, and anterior edge may, for example, contact numbers of teeth other than four, e.g. one, two, three, five or six.

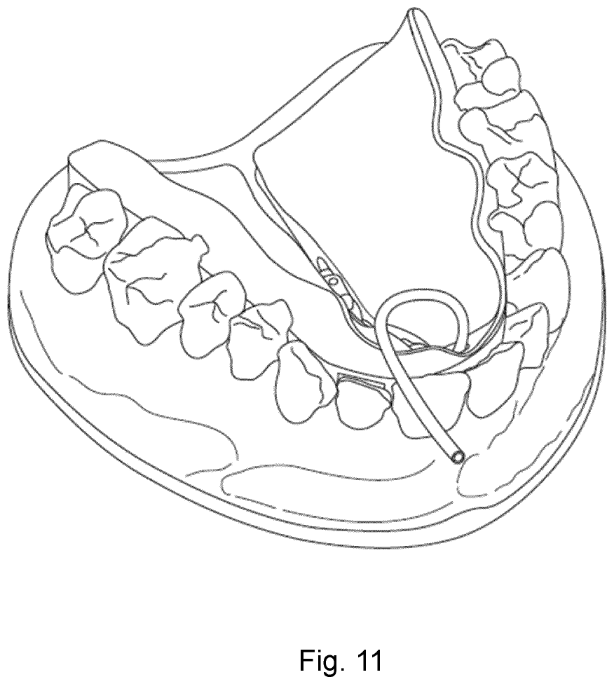

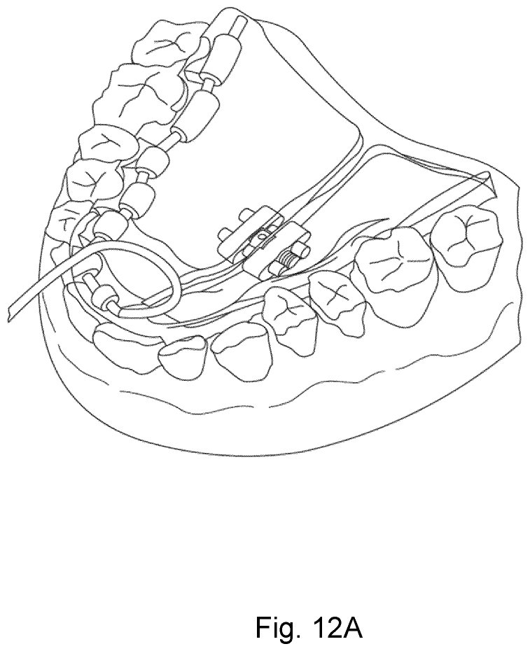

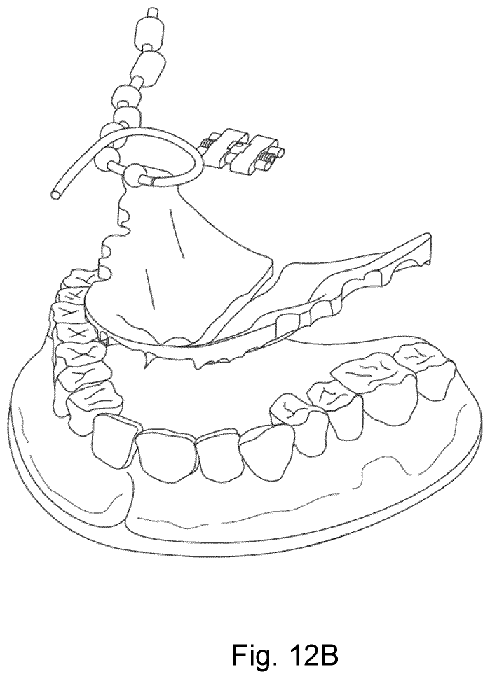

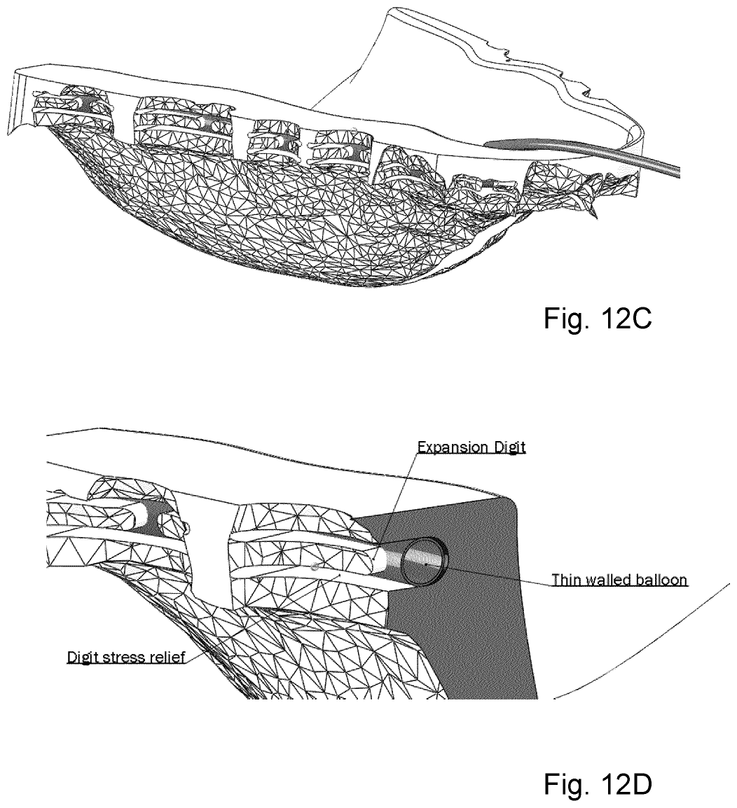

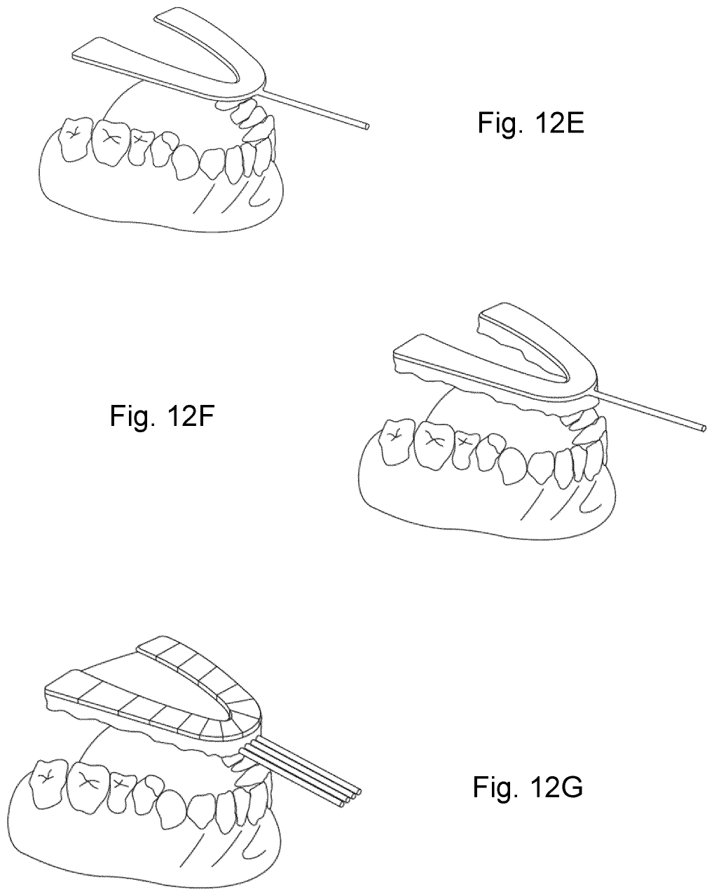

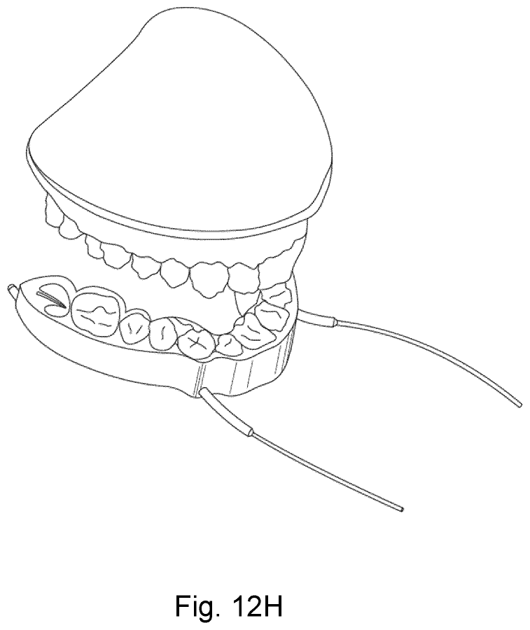

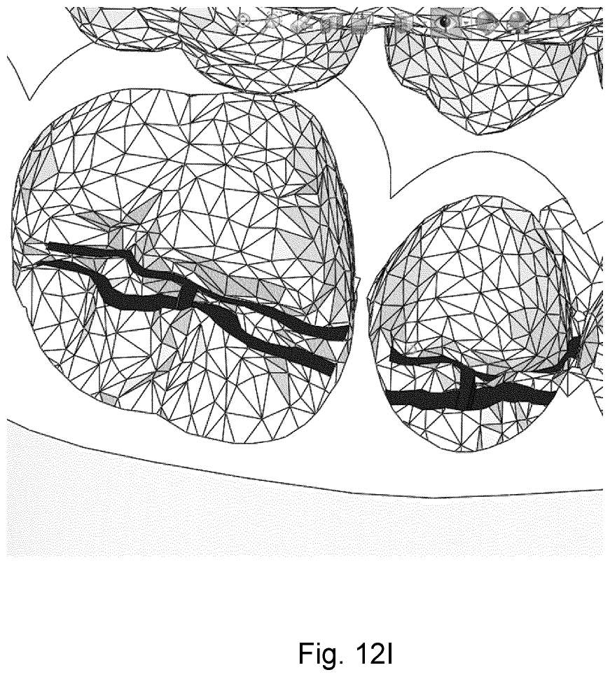

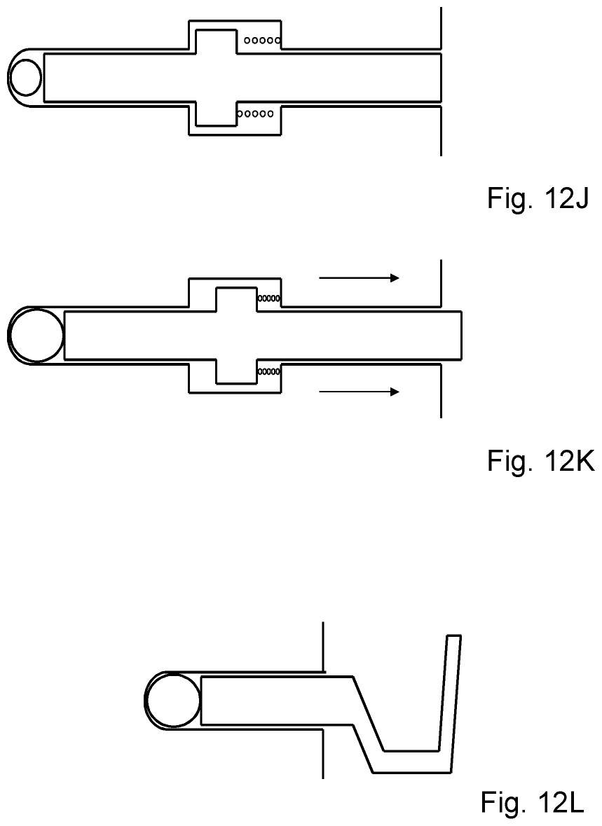

FIGS. 11 to 12B show an embodiment of the present invention, which includes a single moulded plate with a central slot containing a central expansion mechanism, each of which are shaped to conform to the palate, with a central expansion mechanism located therebetween. Alternatively, a similar effect could be achieved using two separate plates. The outer edges of the moulded plates include a series of recesses, the locations of which correspond to the locations of the user's teeth. The device further includes a series of balloons which are configured to fit between the recess and the user's teeth, so that inflation of the balloons causes pressure to be applied to the user's teeth.

FIGS. 12C and 12D show an embodiment of the present invention, which includes a single moulded plate with a central slot containing a central expansion mechanism, each of which are shaped to conform to the palate, with a central expansion mechanism located therebetween. Alternatively, a similar effect could be achieved using two separate plates. The outer edges of the moulded plates include a series of recesses, the locations of which correspond to the locations of the user's teeth. The device further includes a groove or channel in which is housed an elongate inflatable structure, an outer surface of the inflatable structure in contact with an inner surface of a cantilever structure, in the form of a cantilever digit.

The cantilever structure is arranged such that its outer surface is substantially aligned with the tooth facing surface of the recess of the moulded structure. In use, as the inflatable structure is inflated, and so expands outwards, its outer surface exerts pressure on the inner surface of the cantilever structure, causing a tooth facing surface of the cantilever structure to press against the surface of the tooth, thus causing tooth displacement and as multiple teeth are moved—expansion of the dental arch (i.e. maxillary expansion). In this way, the inflatable structure is configured to urge the cantilever structure between an unbiased configuration, (i.e. where the cantilever is stowed within the moulded structure), and a biased configuration in which the cantilever structure extends forward from the tooth facing surface of the recess. In this way, the cantilever structure defines an intermediate structure arranged between the inflatable structure and the tooth.

In an alternative arrangement, the cantilever structure may be arranged to extend out from the tooth facing surface of the recess, even when no force is applied by the inflatable structure. Accordingly, when the moulded plate is installed in the patient's mouth (i.e. such that tooth facing surface of the recess is brought into contact with the tooth), the corresponding tooth exerts a force upon the outer surface of the cantilever structure, which thereby biases it towards the inflated structure. The inflated structure may be configured to resist the resulting force which is exerted upon it by the cantilever structure.

In the embodiments shown of FIGS. 11 to 12D, the inflatable structure may include a plurality of balloons each in fluid communication with each other (advantageous because they can all be inflated from a single source), but alternatively, other embodiments may include multiple balloon strips. The balloons may be independently inflatable, to impart a greater degree of control. A static (i.e. “background”) force may then be applied by adjusting the screw in the central mechanism, so that a constant, outward force is applied to the inner surfaces of the teeth, via the balloons. Then, in use, the balloons may be periodically inflated/deflated in order to provide a periodic component which is superposed onto the static component provided by the moulded plates, in order to apply an always-positive non-zero force to the teeth, in order to effect maxillary expansion. Alternatively the non-zero force can be generated by balloon expansion, further pulsation imparts the periodic component. It is noted that in the embodiments shown in FIGS. 11 to 12B, the balloons are only shown on one side of the device, but the skilled person is well-aware that the balloons could be in place on both sides. For example, there may be a single moulded plate, and the opposite side of the central mechanism may be secured to the palate using a bone screw or other suitable fastener, along the lines of e.g. FIGS. 9 and 10. Alternatively, as in e.g. FIGS. 7A to 8A, the moulded plate with inflatable structure on one side could be combined with an alternative anchor structure on the opposite side, such as being moulded directly to the teeth, or a wire structure. It should be noted that throughout this application, it is envisaged that the first anchor of one embodiment could be combined with the second anchor of another component, where compatible.

FIGS. 12E to 12G show embodiments which are designed to promote remodelling through action at the occlusal plane, which may be defined as an imaginary plane between the upper and lower dental arches. The devices of FIGS. 12E to 12G comprise a continuous U-shaped balloon which is connected to hydraulic tubing, on which the user bites down during use. Then, periodic inflation/deflation of the balloon generates a force which is applied to the user's teeth. A feedback system may be employed to indicate how hard the user should bite. In FIG. 12F, the inflatable bite plate is mounted onto a section moulded to conform to the teeth of the lower dental arch. It is appreciated the reverse or other arrangements are possible. In FIG. 12G, there are a plurality of balloons, each arranged to contact an individual tooth or series/group of teeth, and a plurality of hydraulic tubes, each operably connected with a respective one of the plurality of balloons. The balloons are arranged to conveniently exit the device where the tubing is contained within the device and exits the front of the device. According to an exemplary alternative arrangement the balloons are arranged within a guide element which is configured to prevent the balloon from splaying, or spreading, in a lateral direction as the balloon is compressed between the teeth and the bite plate. The guide element comprises a trough, or channel, having rigid lateral side walls. The channel has an open bottom surface such that, when in use, the balloon is able to make direct contact with the occlusal plane of the teeth. In this way, the rigid walls ensure that the balloon expands in a substantially vertical direction, thereby increasing the application of force upon the teeth. Again other arrangements are possible.

In FIGS. 12H and 12I, cantilever fingers are arranged at the base of the depressions along the occlusal plane. The depressions are moulded to part or all of the crown of the tooth. When the inflatable structure below the fingers expand the cantilever fingers are displaced upwards or an upward angle i.e. upwards but may be slanted. This action supplies force to the uppermost path of the teeth and to the alveolar bone.

FIGS. 12J and 12K show a cross section of an alternative device for maxillary expansion, including an inflatable structure and a sliding member. The sliding member defines an intermediate structure arranged between the inflatable structure and the tooth.

The sliding member is housed within a moulded structure of the device in a similar fashion as described above in relation to the cantilever structures. The device includes a groove or channel in which is housed an elongate inflatable structure, an outer surface of the inflatable structure is arranged in contact with an inner surface of the sliding member. The elongate sliding member is housed in a corresponding channel which extends in a direction that is substantially perpendicular to the longitudinal direction of the elongate inflatable structure. In use, as the inflatable structure is inflated, and so expands outwards, its outer surface exerts pressure on the inner surface of the sliding member, causing it to press against the surface of the tooth, thus causing tooth displacement and as multiple teeth are moved—expansion of the dental arch (i.e. maxillary expansion). The sliding member is shown in FIG. 12J in an unbiased configuration (i.e. a stowed position within the moulded structure) and in FIG. 12K in a biased position in which the sliding member is protruded out from the tooth facing wall of the recess.

The sliding member comprises a locking portion which is wider than the rest of the sliding member. The locking portion is housed in a corresponding portion of the channel, and is configured to limit the travel of the sliding member along the channel. A spring is arranged between the locking portion of the sliding member and an interior wall of the channel locking portion. The spring is configured to resist protrusion of the sliding member from its channel. The locking portion of the sliding member may comprise a substantially square profile when viewed in a longitudinal section, as shown in FIGS. 12J and 12K. Alternatively, the locking portion may comprise a triangular, or truncated, profile when viewed in the same longitudinal section. Thus, the locking portion may taper as it extends away from an outer peripheral surface of the sliding member.

FIG. 12L shows a cross section of an alternative device for maxillary expansion, including an inflatable structure and a ladle shaped member. The ladle shaped member defines an intermediate structure arranged between the inflatable structure and the tooth. The ladle member comprises a ladle, or hook, shaped portion which is arranged to wrap around a patient's tooth, or a dental implant, when in use. In use, the ladle shaped member is actuated in a similar manner to the cantilever member and/or the sliding member described above. In particular, inflation of the inflatable structure leads to the application of force by the ladle shaped portion upon the tooth, causing maxillary expansion. The ladle shaped portion may be configured such that it covers a different proportion of the tooth's labial and lingual surfaces. The ladle shaped portion may be configured so that it does not cover, or make contact with, the occlusal surface of the tooth.

In any of the above described embodiments, a tooth facing surface of the device may be configured to grip the tooth to which it placed in contact with. In particular, at least one of the moulded structure, recess and intermediate structures may be configured with a tooth gripping portion, or tooth gripper. The tooth gripping portion may comprise a rounded or pointed nodule which is protrudes from the tooth facing surface.

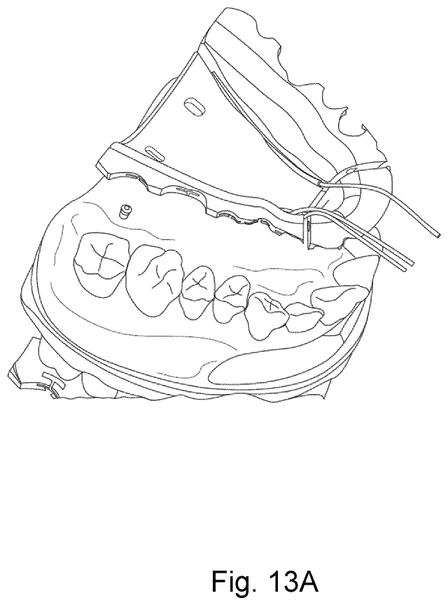

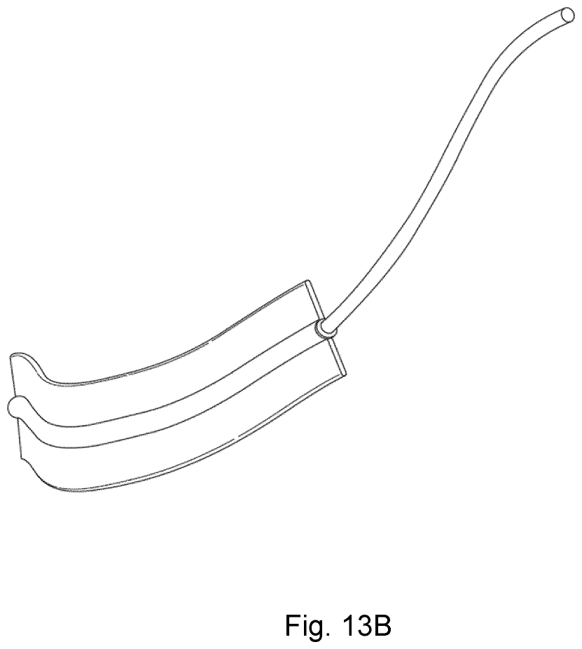

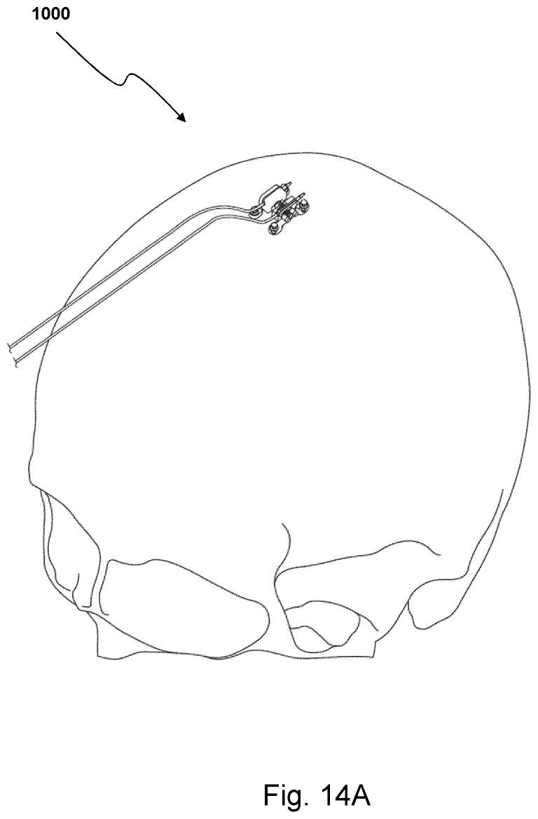

FIGS. 13A and 13B relate to a component in which a central moulded plate may be secured to the roof of a user's mouth, e.g. using bone screws or other suitable fasteners. In addition to the central moulded plate, there are three additional peripheral moulded plates, each shaped to conform to a corresponding surface of the user's mouth, e.g. a portion of the palate and/or an inner surface of the teeth. The three peripheral moulded plates are joined to the central moulded plate via balloons, an example of which is shown in FIG. 13B. The balloons are located between the central moulded plate and the peripheral moulded plates in a manner whereby when the balloon is deflated or only partially inflated with a hydraulic fluid i.e. the joints are articulated facilitating access to the anchoring screw. But when the balloon is inflated, the joint becomes more rigid, and causes each respective peripheral plate to come in to intimate contact with the part of the mouth with which it was moulded from. Activity of the balloons in the tunnels or those between the central and peripheral moulded plates impart force to their respective contacts.

In this way, the balloon may be configured to directly contact the moulded plate structures. An alignment mechanism may be arranged at the joint between the moulded plate structures. The alignment mechanism may be configured to maintain alignment of the moulded plates as they are separated from each other by the expansion of the balloon. The alignment mechanism may comprise at least one alignment rod which is arranged to extend across the channel between the moulded plate structures.

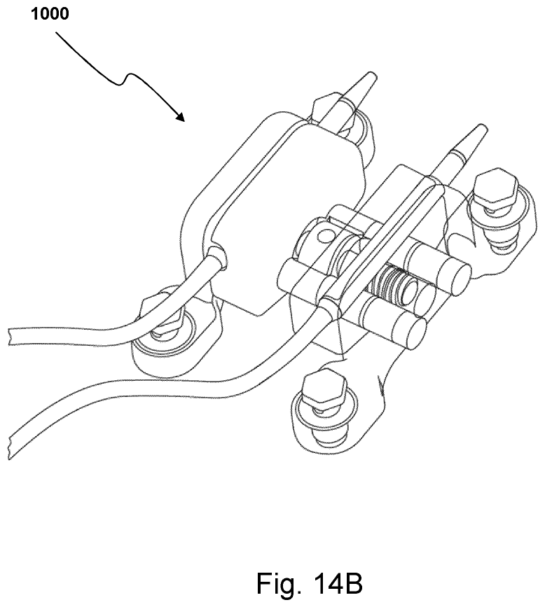

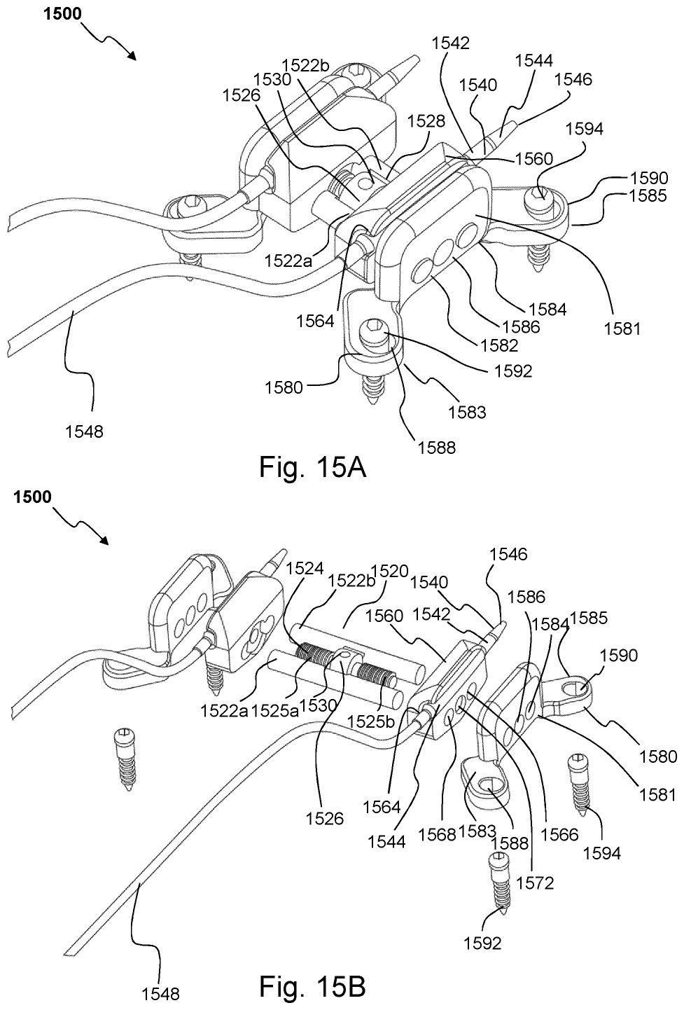

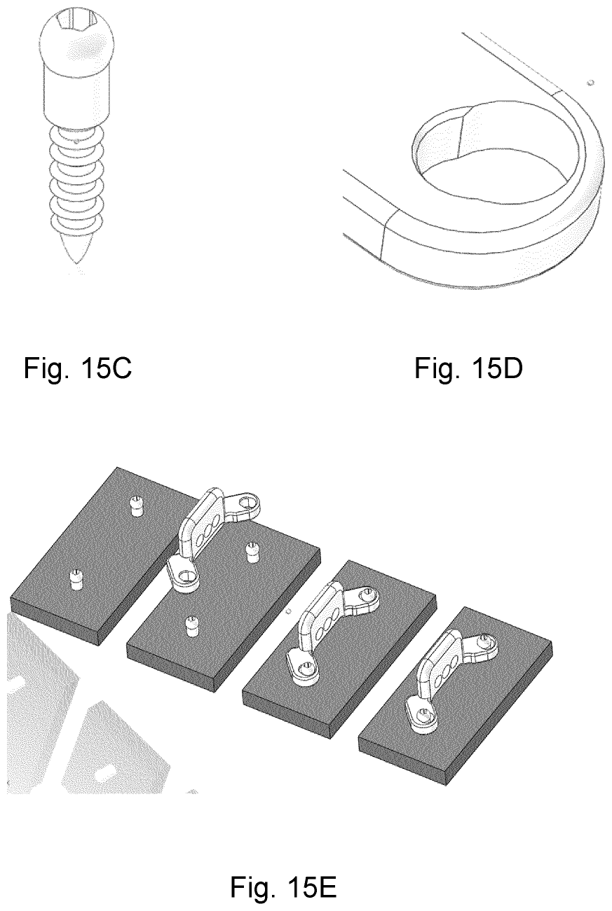

The present invention is not directed solely to devices for oral applications. FIGS. 14A and 14B show an application of a device 1000 according to embodiments of the present invention which is used for distraction of a cranial suture. A number of devices may be arranged along one or more sutures. FIGS. 14A and 14B show example embodiments of a device suitable for this application. FIG. 14A shows the device of FIG. 14B in place on a user's skull. The device 1000 is virtually identical to the device of FIGS. 15A and 15B, so the detailed description will not be repeated here, for the sake of brevity. The devices of FIGS. 14 and 15 differ from each other only in that in FIGS. 14A and 14B the screws have flat hexagonal heads, and in FIGS. 15A and 15B, the screws have domed heads. The operation of the devices are the same. Any of embodiments employing fixation by screws may employ shaped screws e.g. domed and in conjunction with ‘slotted’ screw holes which permit easy exchange of the device without having to interfere with the permanent fixation i.e. in the example given, the screw.

FIGS. 15A and 15B show, respectively, unexploded and exploded examples of components 1500 which may be used as the central expansion mechanism in various embodiments of the present invention. The mechanism 1500 is substantially symmetrical, so we describe only the right-hand side here, for conciseness. Mechanism 1500 includes the following main components, each to be described in more detail in turn: a central component 1520, a hydraulic component 1540, a channel component 1560, and an attachment component 1580.

Central component 1520 includes two elongate cylindrical rods 1522a, 1522b, each having a longitudinal axis extending in a left-right direction. Between the rods 1522a, 1522b, there is a central threaded component 1524 having a threaded portion 1525a, 1525b at each end, the threaded portions having opposite senses. The central region of the threaded component 1524 is not threaded. At the centre of the central component 1520 there is a component 1526. As is best seen in FIG. 15A, each of the rods 1522a, 1522b has a small notch 1528 located at the centre, in which rests the central component 1526. The proximal, unthreaded portion of the threaded component 1524 is integral with component 1526. There is a hollow bore 1530 located in the top surface of the component 1526. A pin may be inserted into the bore 1530 in order to rotate the threaded component 1524 about its longitudinal axis.

The hydraulic component 1540 is comparatively simple. It includes a balloon portion 1542, which is an elongate substantially cylindrical balloon 1544 having a conical or frustoconical tip 1546 at its distal end. The proximal end of the balloon 1544 is connected to a tube 1548, which is connected at its proximal end to a hydraulic pump (not shown). The channel component 1560 is preferably a single piece of material, which includes, on an outside surface a recess forming a channel 1564, the channel shaped to receive the balloon 1544 so that its outer surface is flush against the inner surface of the channel 1564. In preferred embodiments, when the balloon 1544 is in place inside the channel 1564, the outside edge of the balloon 1544 is aligned with or extends pass the outer wall of the component. The channel component 1560 also has two holes 1566, 1568 formed therethrough, each shaped to receive an end of the rods 1522a, 1522b of the central component 1520. There is also a recess located between the two bores 1566, 1568, the recess having a bore 1572 located at its centre, which bore 1572 is configured to receive the threaded end of the threaded component 1524.

The static force is applied by the threaded component 1524 is applied via an internal thread on the central bore of the channel component which is configured to engage with the outer threads on the threaded component 1524.

The attachment component 1580 is also preferably formed from a single piece of material, including three holes 1582, 1584, 1586 which align with the holes 1566, 1568, 1572 on the channel component 1560, and are configured to receive the rods 1522a, 1522b and the threaded end of the threaded component 1524 when they emerge from the outer surface of the channel component 1560. Integrally formed with the main body 1581 of the attachment component 1580 are wings 1583, 1585, each wing extending horizontally from the base 1586 of the main body 1581, and including a hole 1588, 1590, the holes 1588, 1590 being configured to receive a respective screw 1592, 1594. It is often preferred that a fixation pin or pins is permanently fixed to the bone of the skull by means of a bone screw thread. The device to be attached to the fixation pins has a specially formed location and locking slot to match the pin. The fixation pin head is of a spherical nature so as not to offer any sharp edges on which soft tissue may be damaged. In the top surface of the sphere is a hexagonally formed hole so that the bone screw thread attached to the spherical head may be turned with a hexagonal tool. The location slot is formed by two intersecting circles. The larger circle is a little larger than the diameter of the spherical head. The smaller circle is a little larger than the shaft under the spherical head of the fixation pin. The location part of the slot is so sized to allow it to pass over the previously bone mounted location spherically headed pin. The slot is so positioned so that the shaft under the spherical head coincides with the smaller diameter of the slot. In addition, the bottom surface of the spherical head locks against the chamfered edge of the smaller diameter end of the slot. It is all kept together with the compression force from the screw or balloon expansion device. See FIGS. 15C to E.

When assembled, the rods 1522a, 1522b, and threaded end of the threaded component 1524 pass through the bores, 1566, 1568, 1572, 1582, 1584, 1586. This ensures that the components are correctly aligned. The attachment components 1580 are secured to the palate using bone screws 1592, 1594. Then, the component 1500 is adjusted by turning the threads on the threaded component 1524 such that it applies a constant outward force to the palate, via the screws 1592, 1594. This force forms the background static force referred to elsewhere in this application. Then, in use, the balloon 1544 is periodically inflated and deflated using the hydraulic pump (not shown). When the balloon 1544 is inflated, it acts to extend past the plane containing the outer surface of the channel component 1560, and thus applies an additional force onto the inner surface of the attachment component 1580. When this takes place simultaneously, on both sides of the mechanism 1500, the region between the two pairs of screws experiences an extension force which in one application promotes maxillary expansion through separation of the palatal suture.

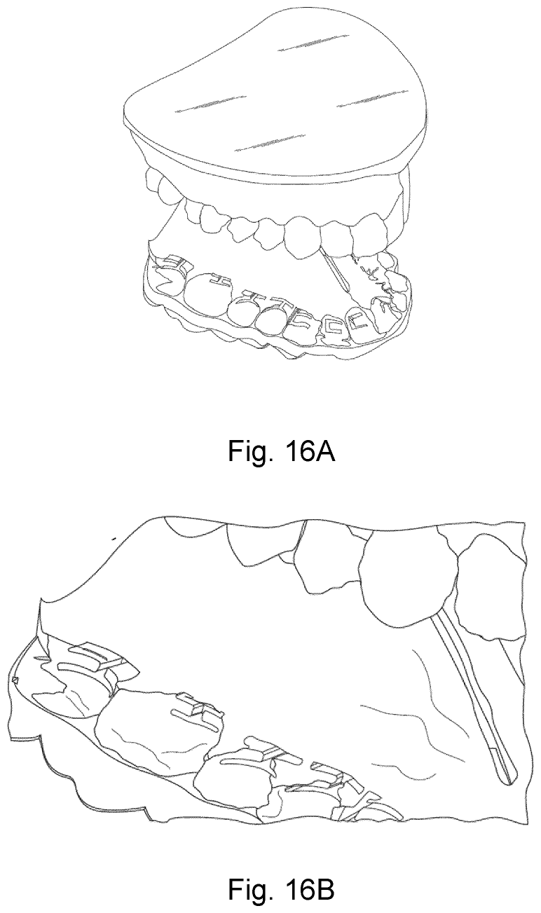

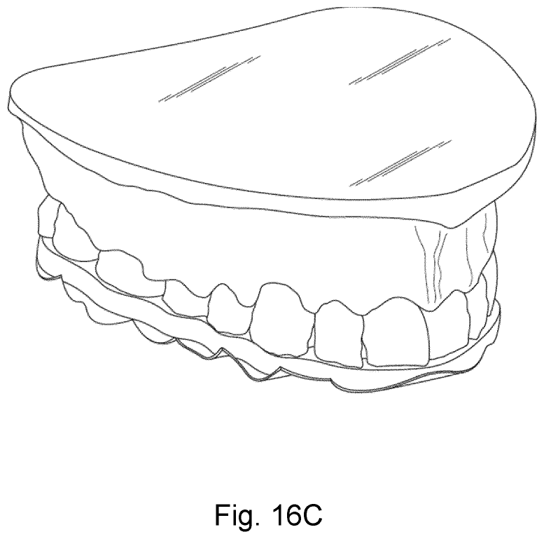

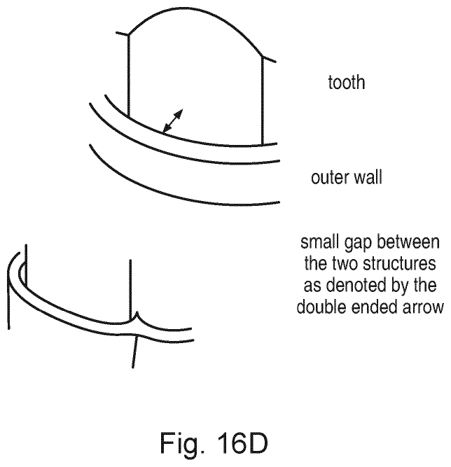

FIGS. 16A to 16D show examples of an insert which may be worn over the teeth in combination with devices according to other embodiments of the present invention, with a view to avoiding, remedying or increasing tooth tilt during restructuring. The insert includes a moulded retainer-like component having recesses which are moulded to conform to the outer surface of the teeth. Each recess/depression is shaped such that the-inner surface of the device is intimate with surface of the tooth, the upper most portions and part of outer surface of the tooth, and there is a structure, in the present case a cantilevered finger, on the corresponding inner surface of the recesses. FIGS. 16C and 16D demonstrate the presence of a small gap between the outer surface of the teeth and the external wall of the device allowing the tooth to move and tip and then be obstructed from further rotation and with further force application begin to upright. The insert is preferably used in combination with the embodiments shown in e.g. FIGS. 11 to 12B, such that balloons expand against the insert, rather than directly against the tooth. The structures in the recesses are preferably located such that as a balloon expands against the insert, the force is transferred to the tooth via the finger, rather than by the whole inner surface of the recess. By ensuring that the cantilevered fingers contact the teeth at an inner surface close to the base of the tooth (i.e. the “gum-end”) it is possible to ensure that the outward force applied to the tooth acts to widen the dental arch through promoting translation of teeth. The embodiment shown in FIGS. 16A to 16D is a maxillary device, but the skilled person will appreciate that mandibular devices are equally feasible.

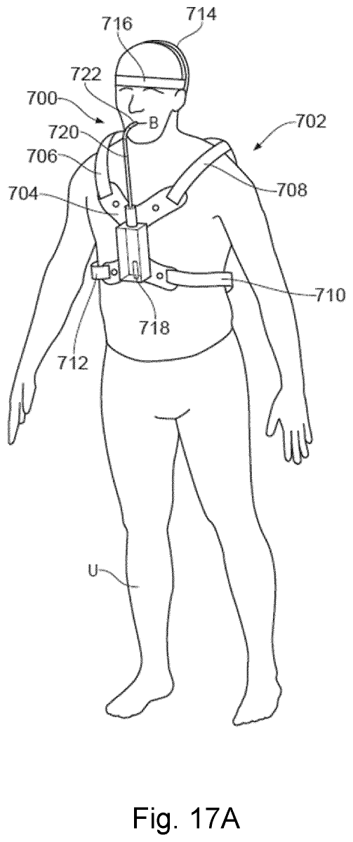

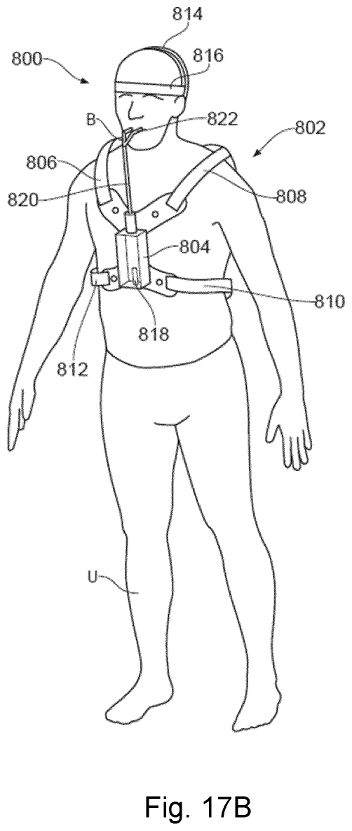

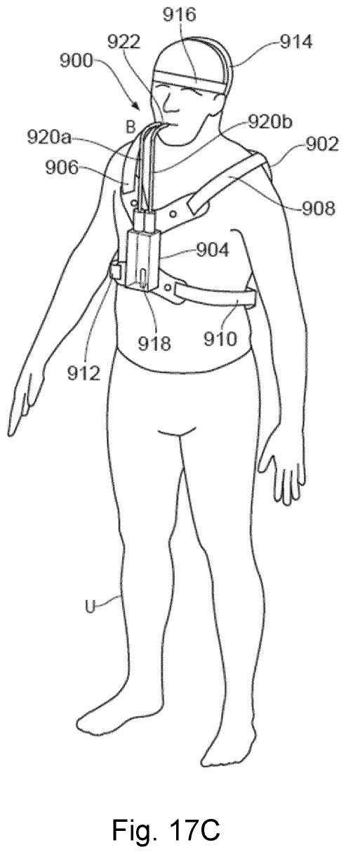

FIGS. 17A to 17C show various ways in which the devices 700, 800, 900 may be mounted onto a user U. It should be noted that the intraoral components of these devices 700, 800, 900 may be as shown in FIGS. 1 to 2, for widening of the maxilla/mandible, or alternatively, and as is described below, they may be used for maxillary or mandibular protraction (moving forward).

The embodiment of FIG. 17A includes harness 702 having a central chest portion 704 having extending therefrom four straps 706, 708, 710, 712. The back of the harness (not shown) includes a single back plate, which is integrally formed with a head plate 714. An additional strap 716 is located across the forehead of the user U to secure the user's head tightly to the head plate 714. The device 700 includes force generator 718 which is configured to generate a periodic linear force within the connector 720. This force may be generated e.g. by a motor or a pump. At the upper end of connector 720, there is a bend B, and a portion 722 enters the user U's mouth. Inside the user's mouth, the rod may be connected to either the maxilla or mandible, and as such may apply a force to that bone as a result of the periodic force, the force acting to cause forward displacement of the maxilla/mandible, thus giving rise to protraction of that bone. The presence of the harness 702 with chest plate 704, back plate and head plate 714 ensures the constant distance between the bend B and the point at which the distal end of the portion 722 contacts the craniofacial feature of interest within the user's mouth, thus maximizing the effect of the force on the craniofacial structure of the user. FIGS. 17B and 17C differ from FIG. 17A respectively in that in FIG. 17B the connector 820 is bifurcated at the bend B, and in that in FIG. 17C, there are two connectors 920a, 920b. These arrangements can help to ensure optimally symmetrical protraction of the desired craniofacial structure.

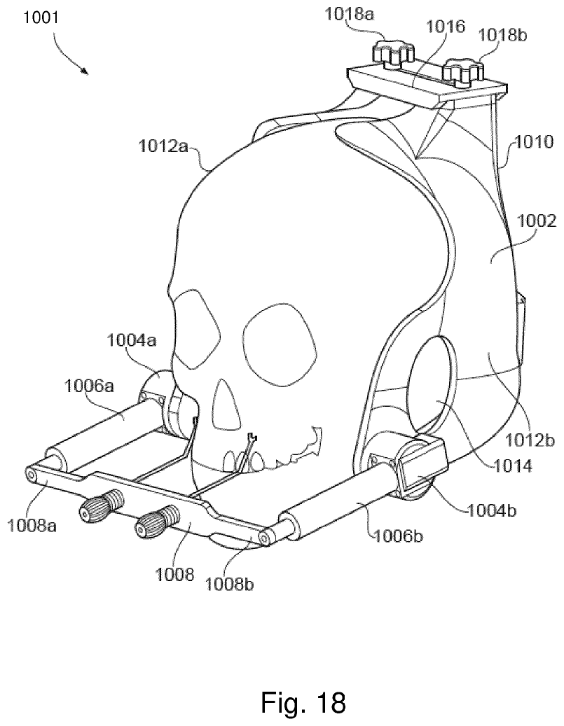

FIG. 18 shows an alternative embodiment of the invention which may be used for maxillary protraction, i.e. drawing the upper jaw forward. The device 1001 includes four main components: a head brace 1002, a pair of motors 1004a, 1004b, a pair of telescopic arms 1006a, 1006b, and a crossbar 1008. The head brace 1002 is shaped to fit snugly onto the back of the head of the user, and extends forward to the base of the lower jaw of the user. The head brace includes a back portion 1010 which contacts the back of the user's head, and two side portions 1012a, 1012b formed integrally with the back portion 1010, and which cover the side of the user's head. The side portions 1012a, 1012b each include a hole 1014 for the user's ear. At the top of the head brace 1002 there is an adjustment device 1016 including screws 1018a, 1018b for adjusting the lateral tightness of the head brace 1002, in order to ensure a secure fit on the user's head. At the front of each of the side portions 1012a, 1012b, there is a respective motor 1004a, 1004b. In the embodiment shown in FIG. 18, the motors 1004a, 1004b are stepper motors, but the skilled person will appreciate that other kinds of motors may be used in practice. Each motor 1004a, 1004b, is connected to the proximal end of a respective telescopic arm 1006a, 1006b, which each act as an extending and retracting actuator. The distal ends of the telescopic arms 1006a, 1006b are connected to the ends 1008a, 1008b of the crossbar 1008.

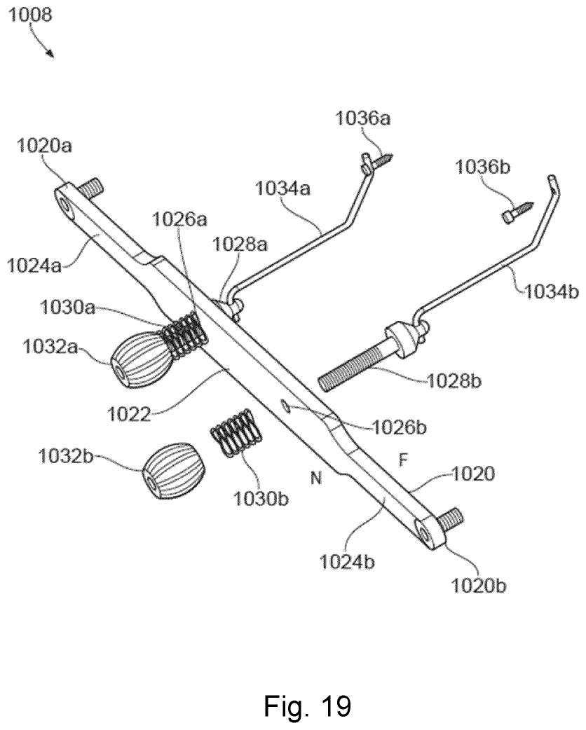

FIG. 19 shows the crossbar 1008 in more detail, in an exploded view. Crossbar 1008 includes an elongate plate 1020 having ends 1020a, 1020b. There is a wide portion 1022 in the central region of the crossbar, and narrower portions 1024a, 1024b either side of the wide portion 1022. Two holes 1026a, 1026b are formed in the wide portion 1022. Zeroing screws 1028a, 1028b are passed through each of the holes 1026a, 1026b. On the near side N of the plate 1020, a wave spring 1030a, 1030b and a tension ferrule 1032a, 1032b are located on the end of the respective screws 1028a, 1028b. On the far side F of the plate 1020, a tension rods or cables 1034a, 1034b are attached to the end of the screws 1028a, 1028b, on the distal ends of which are located bone screws 1036a, 1036b. In order to fit the device, the crossbar 1008 is located in a fully retracted position (i.e. the telescopic arms 1006a, 1006b are fully retracted). Then, then tension rods or cables 1034a, 1034b are attached between bone screws 1036a, 1036b and the screws 1028a, 1028b the attachment mechanism may also be easily reversible e.g. hook or clasp between bone screw and tension rods or cables. The tension ferrules 1032a, 1032b are then turned until a predetermined tension may be felt in the bone screws 1036a, 1036b.

In operation, the motion of the stepper motors 1004a, 1004b is converted into extension/retraction of the telescopic arms 1006a, 1006b, thus causing the crossbar 1008 to move back and forth in front of the user's face. In particular, when the telescopic arms 1006a, 1006b are extended, the plate 1020 is displaced away from the user's face, causing compression of the wave springs 1030a, 1030b, which gives rise to displacement of the screws 1028a, 1028b, thus causing a change in the tension in the bone screws 1036a, 1036b. By selecting a displacement profile of the crossbar 1008 relative to the head brace 1002, caused by the motion of the motors 1004a, 1004b, it is thus possible to construct a tension vs. time profile in the bone screws 1036a, 1036b. Alternatively, or in addition, the telescopic arms 1006a, 1006b (and other components) may be adjusted manually or by an actuating component.

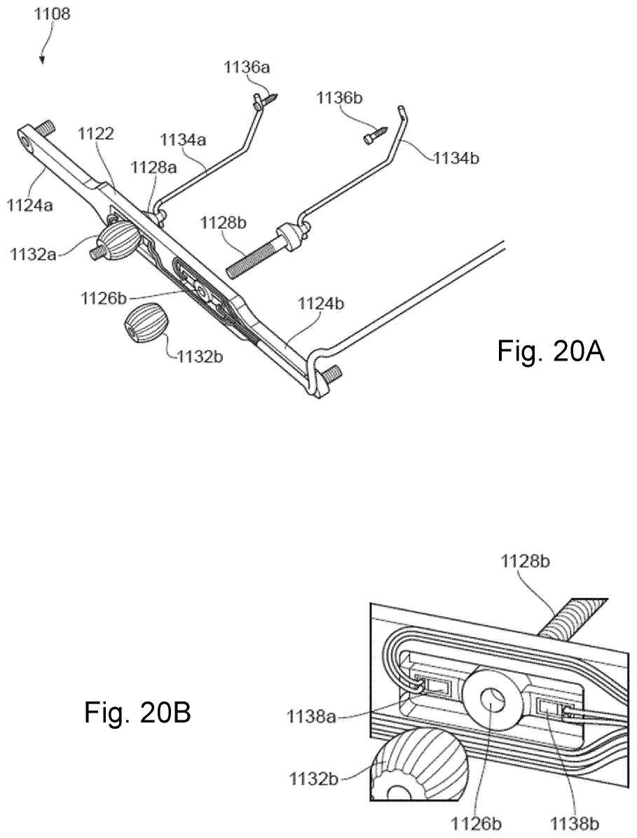

FIGS. 20A and 20B shows an alternative crossbar structure 1108, where like numerals relate to the same features as in FIG. 19. The crossbar 1108 of FIGS. 20A and 20B differs from the crossbar 1008 of FIG. 19 in that it further includes strain gauges 1138a, 1138b for measuring the strain in the tension rods or cables 1134a, 1134b.

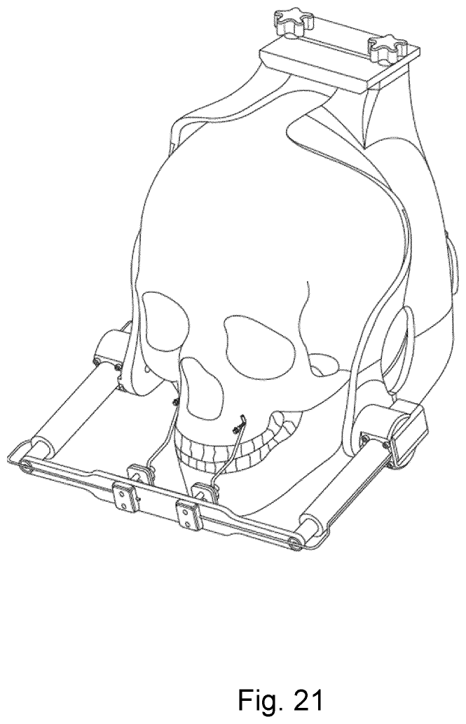

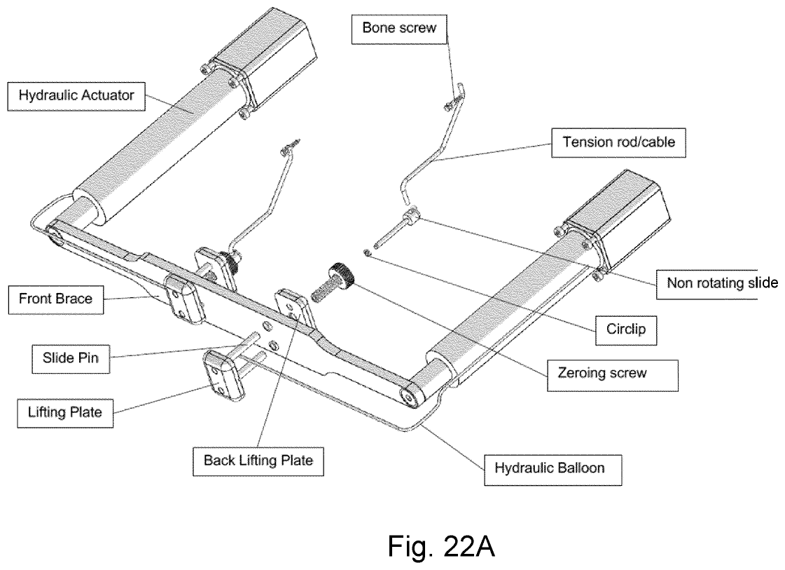

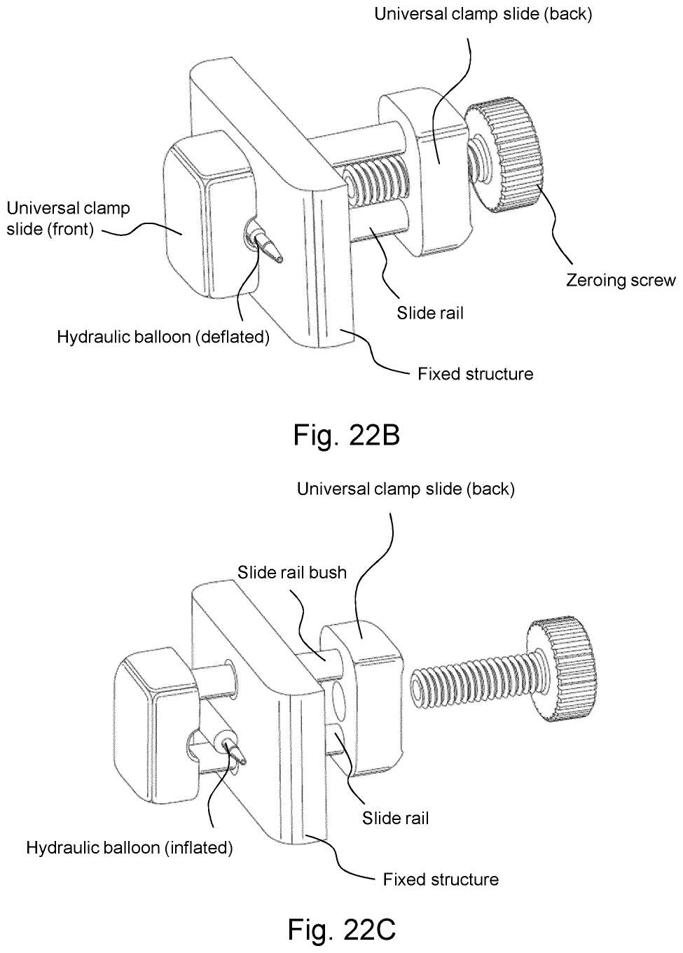

FIG. 21 is similar to the device of FIG. 18, except the actuators are hydraulic pumps, rather than stepper motors. The arrangement is shown in more detail in FIGS. 22A to 22C. The hydraulic balloon acts to increase the distance between the lifting plate and the back lifting plate, thus increasing tension in the tension rod/cable.

FIG. 22A shows an exploded view of the crossbar structure. The operation of the structure is as follows:
The hydraulic actuator is in a fully retracted position, and the hydraulic balloon is deflated.
The tension rod/cable is attached between the bone screw and the non-rotating slide.
The zeroing screw is then turned until there is a tension felt in the bone screw.
At this point, the lifting plate is flush against the front brace.
Now, the apparatus is in the “rest position”.
Coarse oscillation can be achieved by extending and retracting the hydraulic actuator.
Pressure changes in the hydraulic actuator provide feedback information on loading the bone. This provides in one instance low frequency underlying oscillating forces.
Pressure changes in the hydraulic balloon displace the lifting plate forward, thereby applying a load to the bone. Pressure changes in hydraulic balloon also provide feedback information on loading bone. This is used for small high frequency pulsating force.

Each of the hydraulically actuated head brace arrangements, as described herein, may be configured with at least one detachable self-sealing connection. The self-sealing connection may include, for example, at least one of a one-way valve and a reversible valve. The self-sealing connections are configured to enable the hydraulic pump to be reversibly connected, to one or more hydraulic balloons without effecting the hydraulic pressure in those balloons.

FIGS. 22B and 22C show the lifting plate assemblies in more detail, and may be described as follows:
The universal clamp slide front and back are connected together via two parallel slide rails that slide freely through two slide rail bushes.
The bushes are fixed to the fixed structure
The zeroing screw connects to the rod or cable connected to the skull
In the deflated state, the front universal clamp slide (front) is in contact with the vertical surface of the fixed structure. This is the at rest state.
When the hydraulic balloon is inflated, the Universal clamp slide (front) is forced away from the vertical fixed surface.
The direction will be towards the left direction and causes the zeroing screw to create tension in the rod or cable connected to the skull.
By varying the volume of hydraulic fluid in the hydraulic balloon will cause the distance between the Universal clamp slide (front) and fixed structure tension in the rod or cable to vary and so the applied tension in the cable or rod connected to the skull.
To create compression on the skull via a rod, the Universal Clamp slide (back) will have to be pushed to the right-hand direction. This can be achieved by positioning the hydraulic balloon to the right of the fixed structure and inflating the hydraulic balloon, forcing the universal clamp slide (back) away from the fixed structure and pushing the zeroing screw to the right hand side.

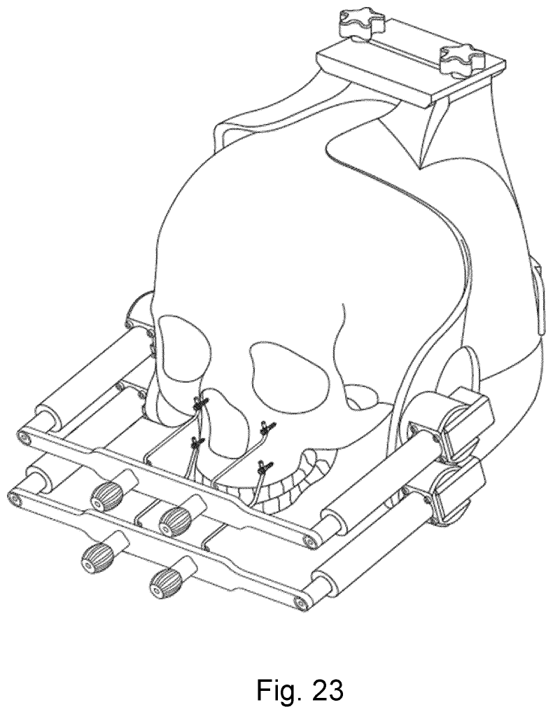

FIG. 23 shows a similar device to the device of FIG. 18, except there are two set of stepper motors, and two crossbars. In this way, a protraction force can be applied evenly across a vertical extent of the cranium.

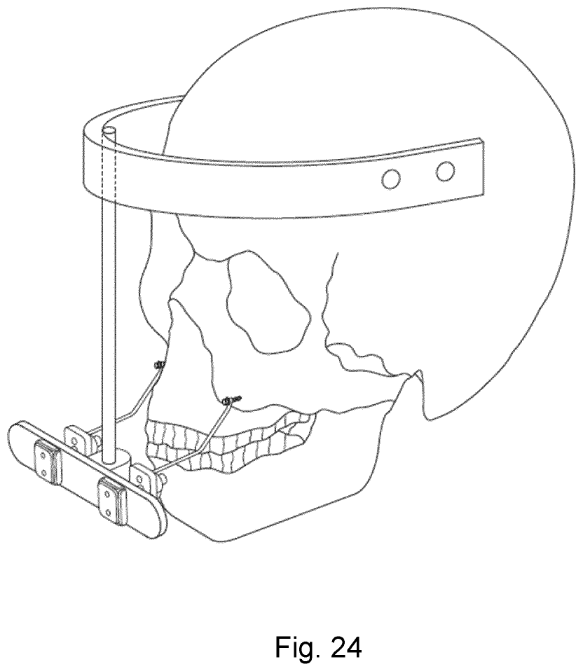

FIG. 24 shows an alternative arrangement in which a rigid arcuate support is fixed to the skull using bone screws or other suitable fasteners. A rigid central stem is attached to the centre of the rigid arcuate support, and the other end of that step is attached to a crossbar like the crossbar in e.g. FIGS. 19 to 20B. This device operates in a similar manner to the device of e.g. FIG. 21, other than the fact that the device is secured to the skull by the bone screws, rather than by the friction between the user's head and the inner surfaces of the head support structure. The corrective forces which are applied by this device are generated by an actuator, as described above. The actuator may comprise a hydraulic pump or a stepper motor, and may be mounted to the rigid arcuate support. The actuator is removable mounted to the arcuate support. Furthermore, the crossbar is also removable from the wider structural arrangement.

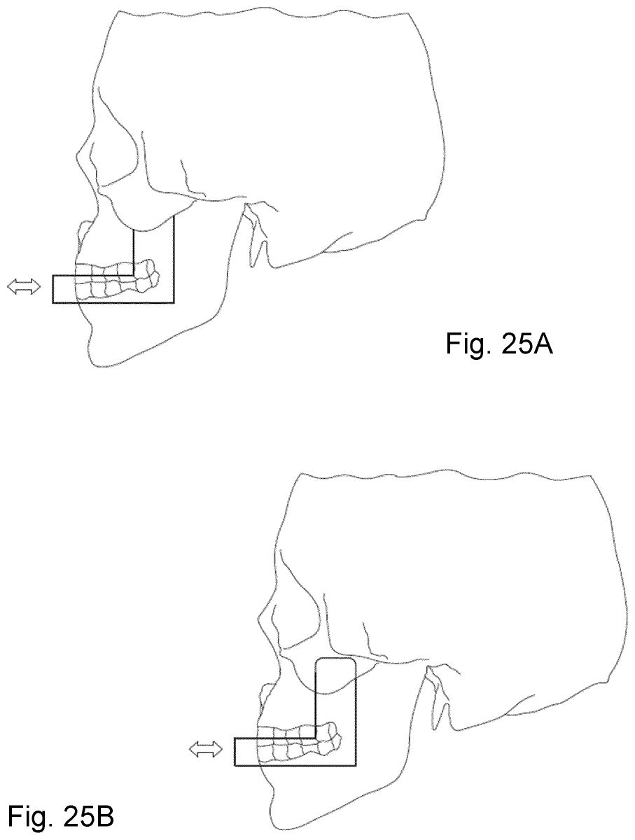

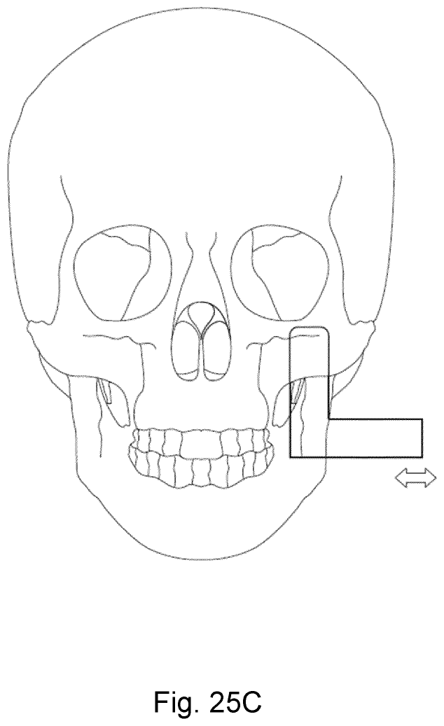

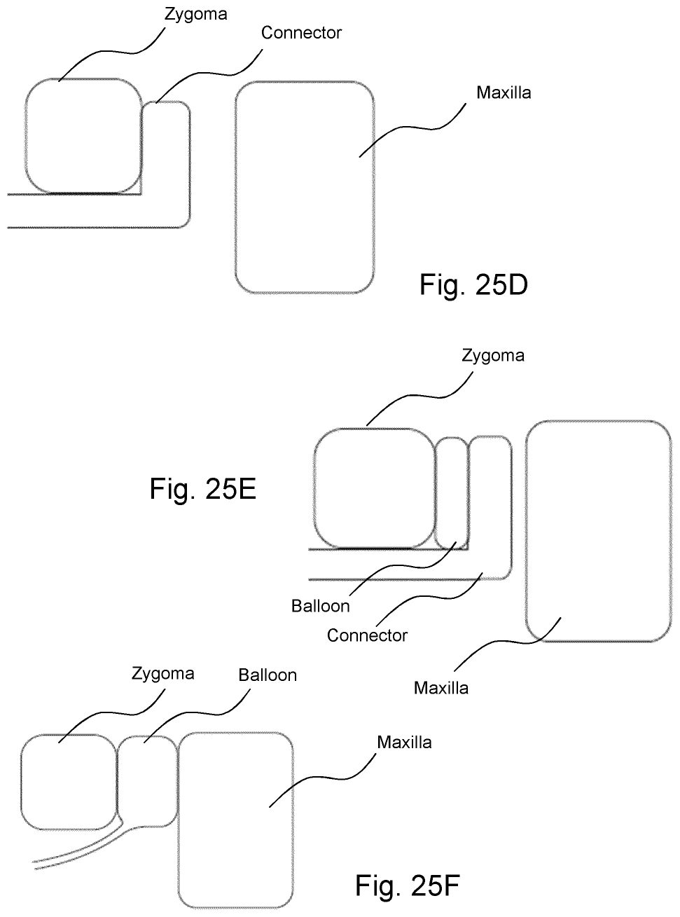

FIGS. 25A to 25F are schematic representations of an embodiment in which force is applied to the space between the zygoma and maxilla. In the embodiments shown, an L-shaped connector is employed, the outer end of which (i.e. the end which is not in contact with the cranium) may be attached to a device such as those shown in any of FIGS. 17A to 17C, 18, 21, 23, 24, 26, 29A, 30, 31A, 32A, 34A . In FIGS. 25A and 25B, the L-shaped connector is configured for maxillary protraction, and in FIG. 25C, the L-shaped connector is configured for maxillary expansion. It should be noted that although the connector in FIGS. 25D to 25F is shown as a sharp L-shape, in preferred embodiments of the invention, the connector would be shaped to the contours of the bone surfaces in question as well as allowing for any occupancy by inflatable structures. FIGS. 25D to 25F illustrate three different ways in which a force may be transferred to the relevant cranial structure:
In FIG. 25D, a rigid connector is placed between the zygoma and the maxilla, and force transferred from the force generator to the connector is transmitted directly to the zygoma, to promote bone growth and/or bone displacement.
In FIG. 25E, a balloon is in place between the connector and the zygoma. The connector is fixed in place and may be applying a constant force to the bone. Force is transferred to the zygoma by periodic inflation and deflation of the balloon. In some embodiments, the balloon may be inflated/deflated dynamically to provide an “active cushion” effect as the connector actuates.
In FIG. 25F, there is no rigid connector, and a regular or shaped balloon fills the space between the maxilla and the zygoma. In such embodiments, as the balloon inflates/deflates periodically, the zygoma is displaced laterally (because it is less resistant to movement than the maxilla). Any adverse compression of the maxilla can be overcome by using e.g. the arrangement of FIGS. 25D or 25E inside the palate, or by using another intraoral device such as those described elsewhere in this patent application.

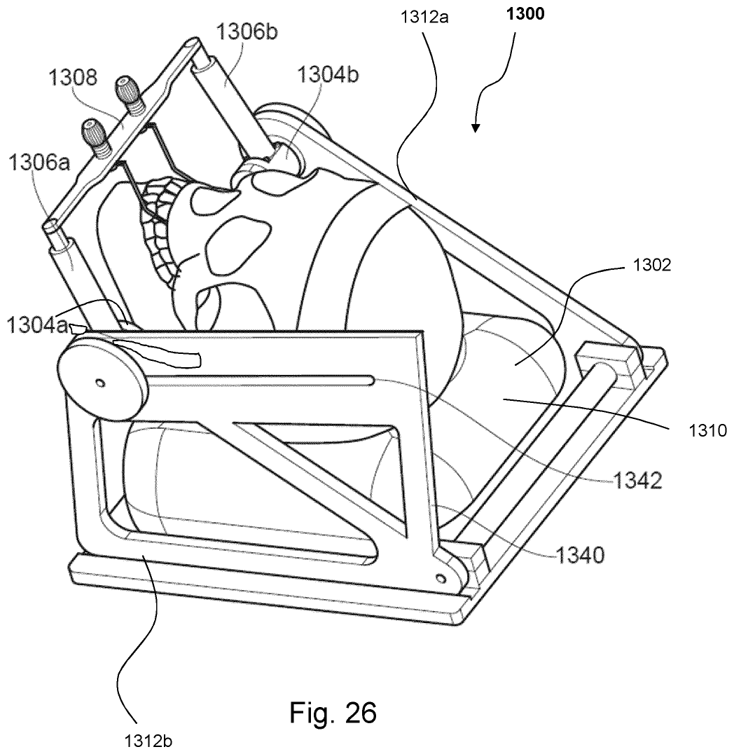

The device 1300 of FIG. 26 is similar to the devices shown in e.g. FIG. 18, but is for use when the user is lying horizontal, rather than sitting or standing upright. Similar reference numerals are used below to represent similar structures, as will be appreciated. Device 1300 includes a head support 1302, motors 1304a, 1304b, telescopic arms 1306a, 1306b, and a crossbar 1308. The head support 1302 includes a headrest 1310 including a cavity for receiving the head, and side portions 1312a, 1312b. In “lying-down” devices such as those shown in FIG. 26, the weight of the user's head acts to prevent the user's head from moving forward during the application of the periodic force.

The head brace 1302 optionally further includes additional frame elements 1340 (only one is shown, but the skilled person understands that there could be one on each side, or none at all), which are formed integrally with the side portions 1312a, 1312b. Each frame element 1340 includes a slot 1342, into which a corresponding protrusion on the motors 1304a, 1304b fits. The operation of device 1300 is the same as that of the earlier devices but in a different orientation.

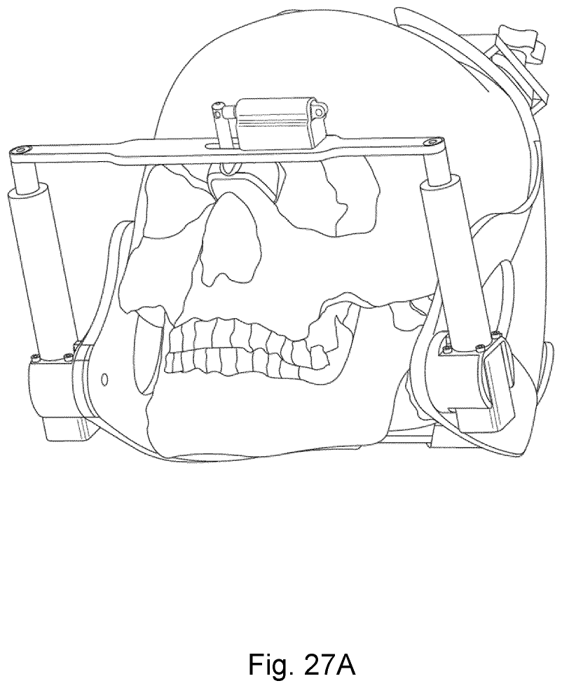

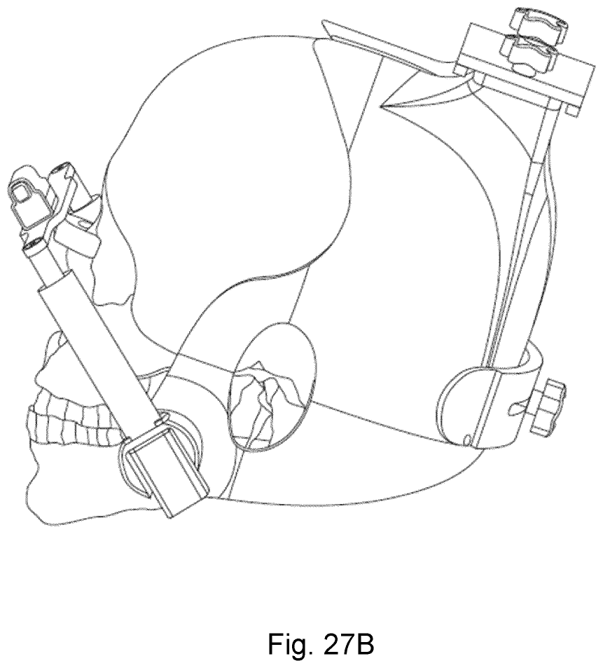

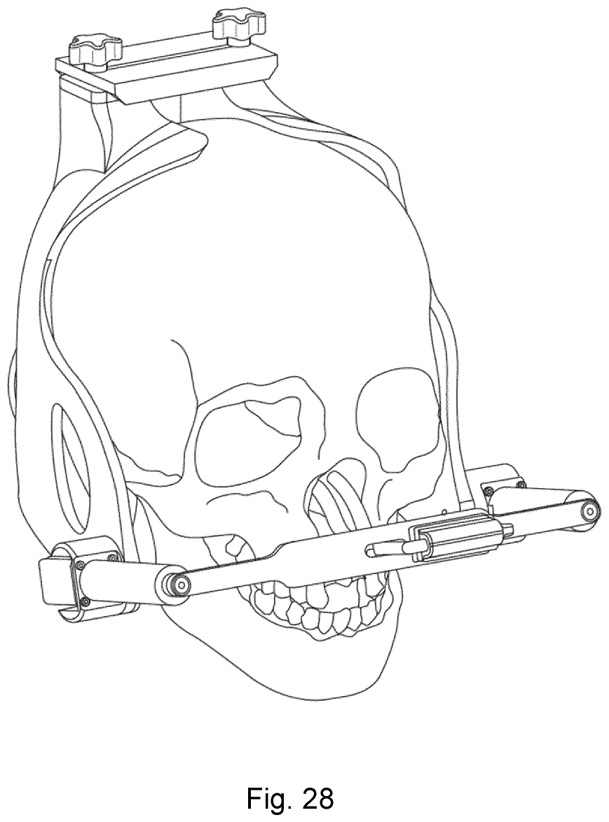

FIGS. 27A to 27B show alternative examples in which it is possible to rotate the connectors between the crossbar and the actuators, e.g. as is shown, to connect the tension rods to the intranasal structures. The device shown in FIG. 28A is arranged for restructuring of proximal cranial structures including the ethmoid bone. In an alternative arrangement, the head brace is configured such that the attachment point is provided at the cheek bones or zygomas'. The frame elements and motors are configured to generate an expansive force, which is applied to the cheek bones in a substantially upward direction. It is recognised that whilst the force that is exerted by the frame elements is expansive, the patient would experience a compressive force.

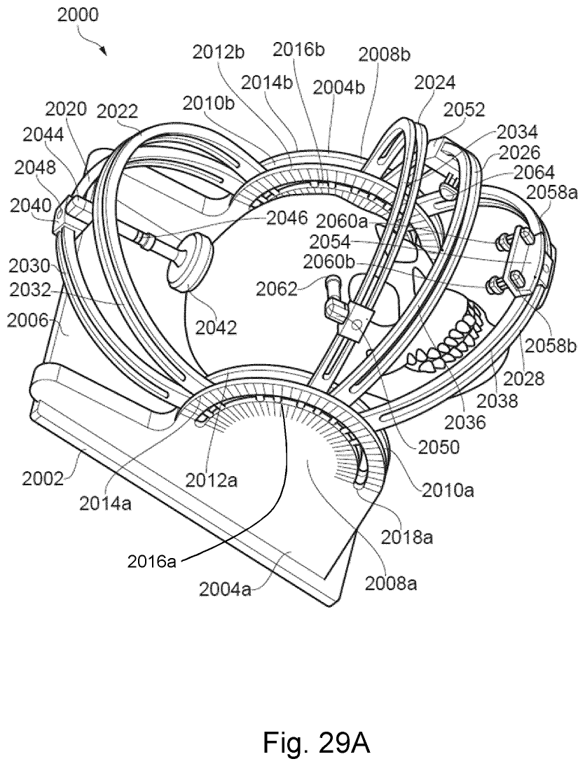

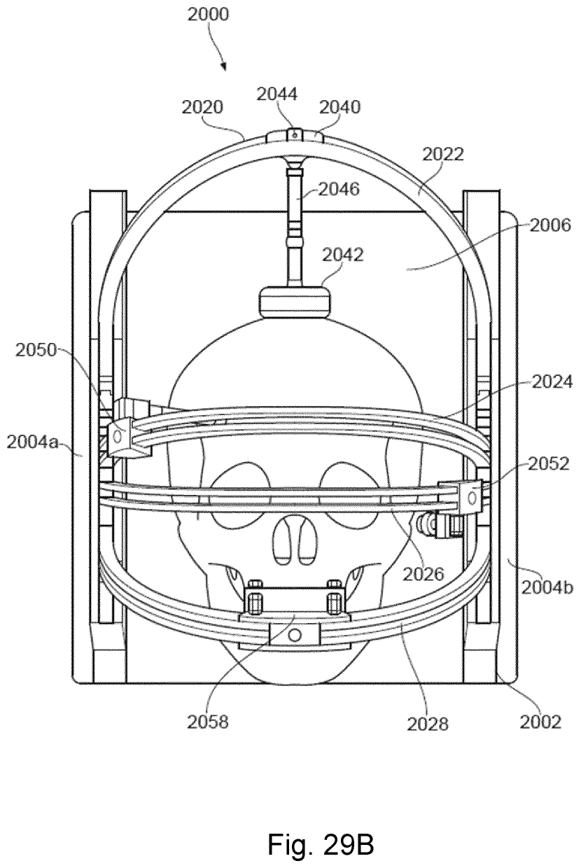

FIGS. 29A and 29B show an alternative embodiment of the device which is able to apply a more diverse range of periodic forces to the user's craniofacial structures. The device 2000 includes a main portion 2002 including side portions 2004a, 2004b mounted on base 2006. Each side portion 2004a, 2004b includes a semi-circular portion 2008a, 2008b including a semi-circular recess 2010a, 2010b, the recess 2010a, 2010b being defined by inner wall 2012a, 2012b, and outer wall 2014a, 2014b. The inner walls 2012a, 2012b each include a slot 2016a, 2016b, and the outer walls 2014a, 2014b each include a slot e.g. 2018a. The space between side portions 2004a, 2004b is approximately the width of a human head.

Device 2000 further includes five arcuate rails 2020, 2022, 2024, 2026, 2028. It will be appreciated by the skilled person that other embodiments of the invention can exist having fewer (i.e. one, two, three or four) rails, or more rails. In the embodiment shown, each of the arcuate rails 2020, 2022, 2024, 2026, 2028 are semi-circular, but other arcuate shapes may be used equivalently. Each end of each arcuate rail 2020, 2022, 2024, 2026, 2028 is located within a respective one of the semi-circular recesses 2010a, 2010b, and has protrusions extending through each of the inner slots 2016a, 2016b, and outer slots e.g. 2018a.

Each of the arcuate rails 2020, 2022, 2024, 2026, 2028 includes a mounting groove or slot 2030, 2032, 2034, 2036, 2038 running along most or all of its length. Generally, these slots 2030, 2032, 2034, 2036, 2038 are for mounting a component on the arcuate rail in question, which could be the force generator, force transmitter (or both), or the anchor.

In FIG. 29A, it is clear that each of arcuate rails 2020, 2024, 2026, and 2028 each have a different attachment. Mounted on rail 2020 is a vibrating plate assembly 2040. The vibrating plate assembly includes a vibrating plate 2042, a force generator 2044, a rod 2046 (which could be telescopic) and a mount 2048. The force generator 2044 is mounted to the rail 2020 via mount 2048. The output of the force generator 2044 is transmitted to the rod 2046, and then to the vibrating plate 2042. The vibrating plate in this case is flat, but in some alternative embodiments, the plate may be shaped to fit various craniofacial structures. It is to be understood that though the embodiment of FIG. 29A shows only a single rail 2020 having a vibrating plate assembly 2040, other embodiments may have additional vibrating plate assemblies. In implementations where, for example, an upward force is being applied by one or more force transmitters, the plate may not vibrate, and may act as an anchor restricting movement of the head in response to the applied periodic forces, thus maximizing the effect of the force.

Rails 2024, 2026, 2028 each have force transmitting components 2050, 2052, 2054 on them. The structure 2054 on rail 2028 is equivalent to the crossbars 1008, 1108, 1308 described earlier in this application. It differs in that the equivalent feature to the plate 1020 is slightly curved in order to be able to slide along the rail 2028 without resistance. The assembly 2054 is configured to apply a tension force to a given craniofacial structure, and includes force generators 2058a, 2058b, force transmitters 2060a, 2060b, and may have a tension rod or cable (not shown) connected thereto. Similarly assembly 2052 is configured to apply a tension force to a given cranial structure, and includes force generator 2064 and may have a tension rod or cable connected thereto.

Components 2050 and 2052 are compressive force transmitters which operate in the same way as the vibrating plate assembly 2040, but have different shaped (i.e. smaller) plate 2062.

In the embodiment shown in FIG. 29A, the component 2050 may be able to rotate relative to the rail 2024 in order to alter the direction in which it is able to apply the compressive force.

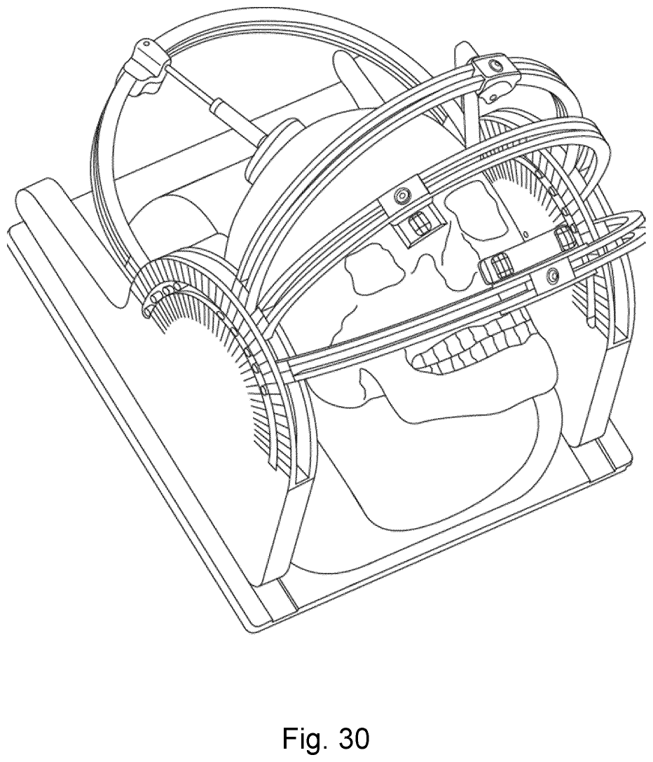

FIG. 30 shows a similar embodiment to FIG. 29A with a head cushion installed on the base.

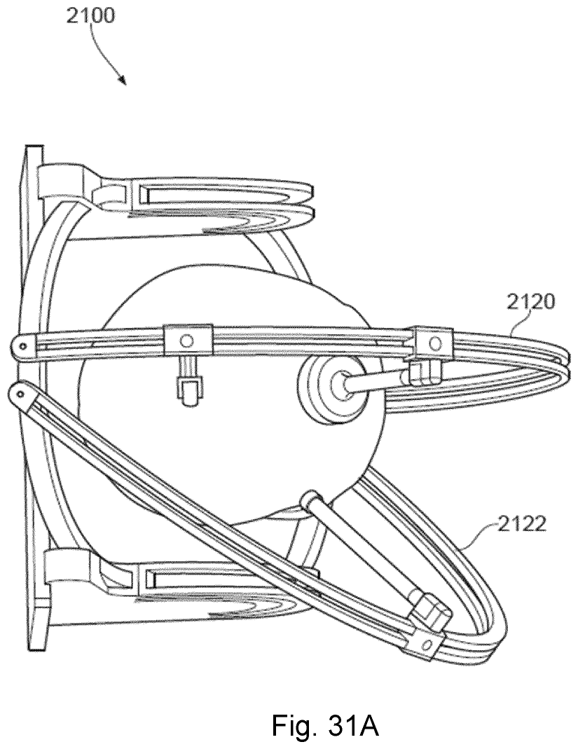

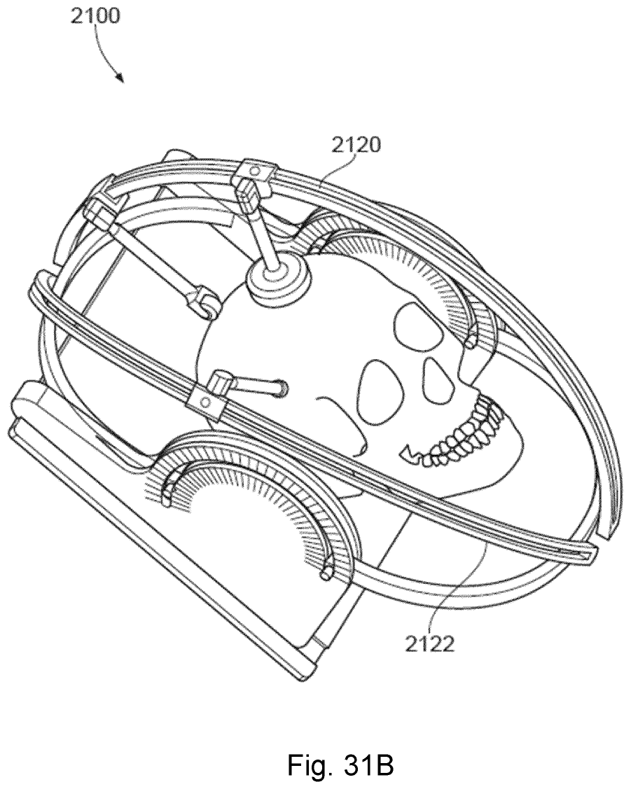

FIGS. 31A and 31B show a similar device to the device of FIGS. 29A and 29B, except the rails 2120, 2122 are oriented vertically, rather than horizontally, and therefore are able to rotate about the user's head from left to right, rather than from top to bottom. The components located on the rails are able to move along their respective rails in an up and down directions. It should also be noted that one of the rails includes a roller. The roller is not limited to this embodiment, and could feature in any embodiment of the invention.

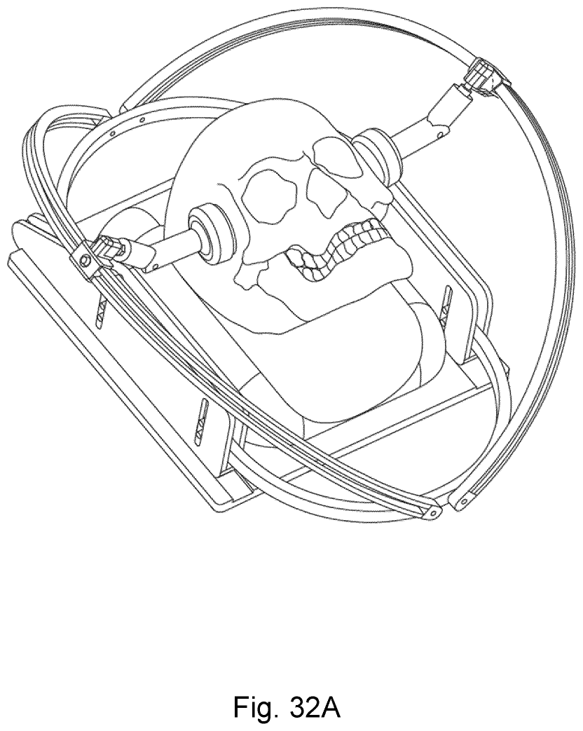

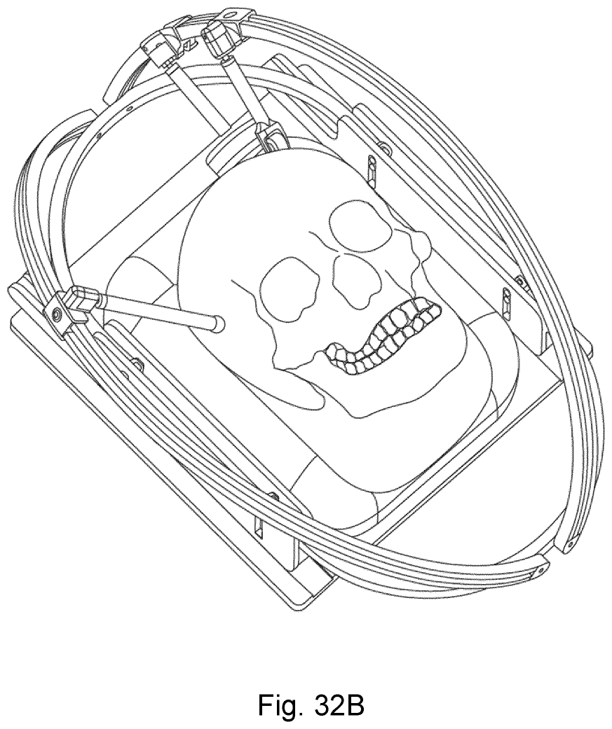

FIGS. 32A and 32B shows modified versions of devices having vertical rails in which the walls are removed in order to reduce restriction of movement in the lateral direction. This example demonstrates a frame work being used to position two opposing pressure pads. These pressure pads are positioned across the skull. The actuators may apply a variable force to the surface of the skull. In addition the pressure pads are able to oscillate about its axis to apply torsion to the skull surface. These oscillations may be restricted to +/−45°, e.g. to push downwards with a quarter turn. These oscillations may be uniformly sinusoidal or random

In one example one pressure pad may apply an inward and rotational force and the other side may apply no or a constant oppositional force to restrict head movement

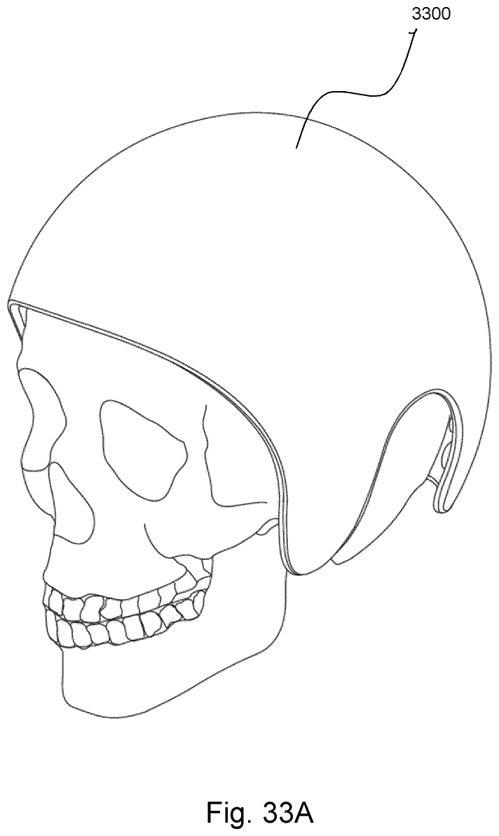

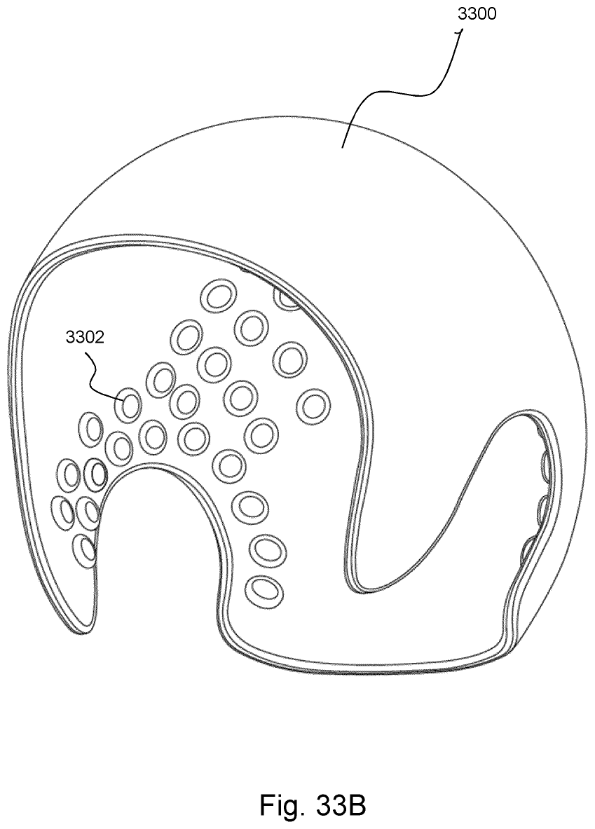

FIGS. 33A and 33B show a cranial restructuring helmet 3300 according to an embodiment of the third aspect of the invention. The inner surface of the helmet 3300 is shaped to conform snugly to the outer surface of the user's head. As seen in FIG. 33B, the inner surface of the helmet 3300 includes a plurality of columnar balloons 3302. Although FIG. 33B shows the balloons 3302 only covering part of the inner surface of the helmet 3300, the skilled person will appreciate that the present invention covers embodiments in which any amount of the inner surface of the helmet 3300 is covered with annular 3302. The skilled person will also note that the balloons need not be annular. In use, the two anchor points may correspond to two points on the user's cranium which are opposite to each other, and the two anchors of the cranial restructuring device correspond to the portions of the inner surface of the helmet 3300 which contact those anchor points. A periodic force may be applied to the cranium by periodic inflation and deflation of the balloons 3302. In preferred embodiments, the helmet 3300 is sized to fit tightly onto the user's head, in order to provide a “background” compression force, which is then supplemented by the periodic force which is provided by inflation and deflation of the balloons 3302. The balloons may be connected individually directly to the hydraulic compressor, or they may be connected as a daisy chain so that a whole block of balloons will inflate simultaneously. With the hydraulic balloons deflated the helmet is placed over the head and the balloons inflated. The balloons pressure may be varied to any waveform desired to create a pulsating massaging effect. With the correct interconnection of balloons and multiple pressure lines to the compressor, the balloons when inflated/deflated sequentially may effect an undulating massaging sequence. It is noted that in this, and in all other embodiments of the invention, there may be a plurality of balloons, which may of different shapes, sizes, orientations, and positions (including relative proximity to skull. The balloons could also be different textures. There could be additional elements which could displace inflated balloons, or equally well the inflated balloons could displace additional elements.

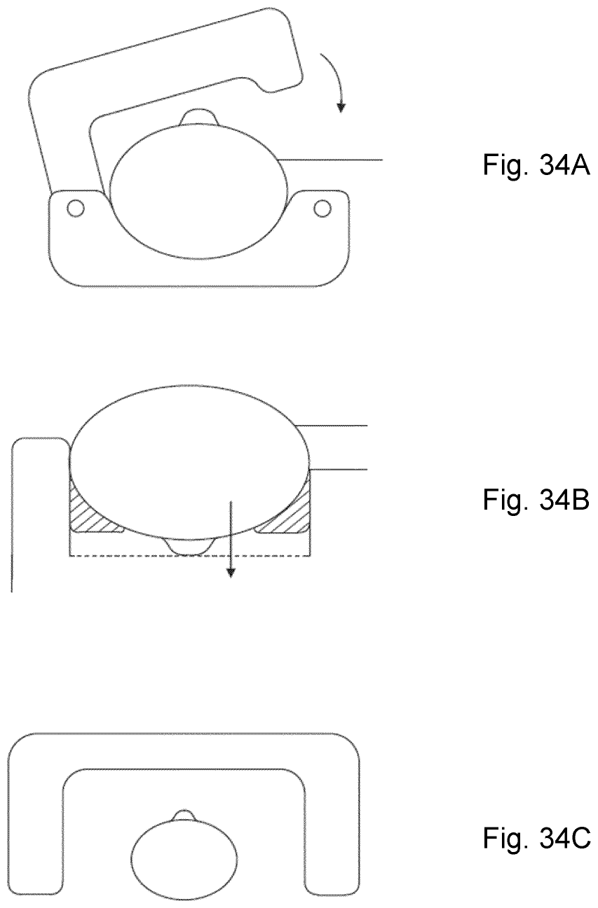

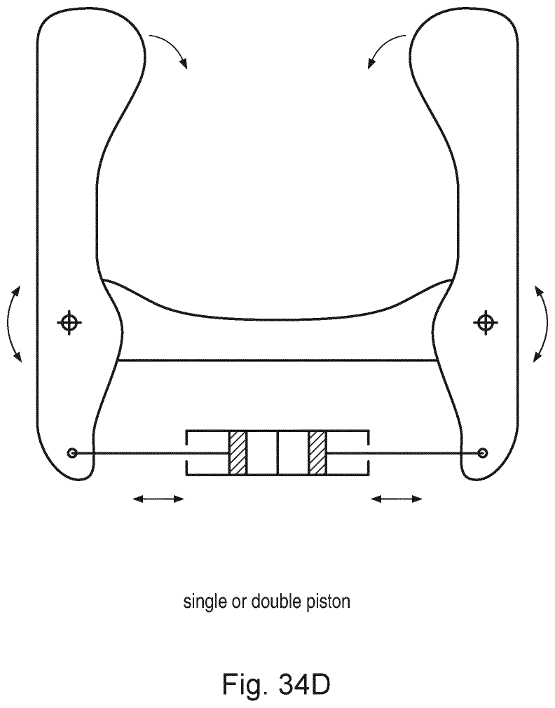

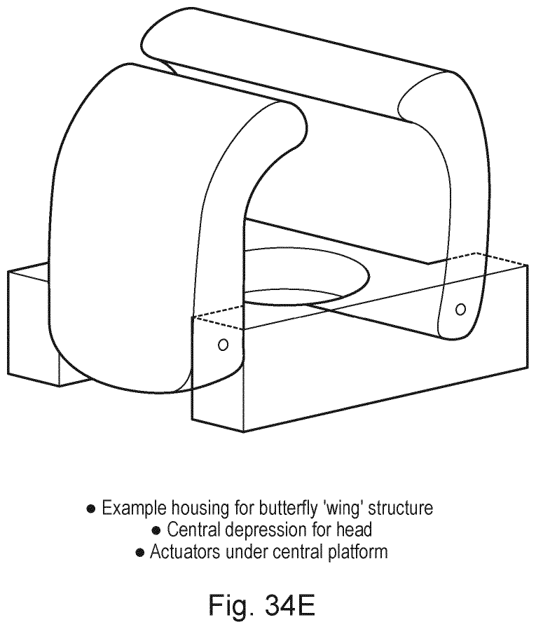

FIGS. 34A to 34C are simplified diagrams of an alternative cranial devices. In FIG. 34A, the user places their head in the lower seat, which includes a recess to allow it to better conform to the shape of the user's head. Then, the upper portion may be lowered over the user's face. The device is preferably configured to fit snugly over the user's face in order to provide a background force. Then, a periodic force may be applied hydraulically, e.g. using annular balloons as in FIGS. 33A and 33B. Alternatively the upper portion may have other components to apply compression or tensioning forces such as the compressive force transmitters shown in FIGS. 29A and 31 and 32B FIGS. 34B and 34C demonstrate other configurations of devices in which devices according to the present invention might be mounted.

FIGS. 34D and 34E show a similar embodiment having a “butterfly” arrangement, in which two side portions are brought together inwards across a user's face.

The embodiments shown in FIGS. 34A to 34E may be modified to provide tension forces to a cranial structure rather than compressive forces. For example, an attachment portion located on an inner surface of the device may be arranged to connect to e.g. bone screw or other fixture on the user's cranium, in a manner whereby tension is applied to the fixture. This would then represent the background force, and a periodic force may be applied hydraulically on top of this, in the same manner as other embodiments.

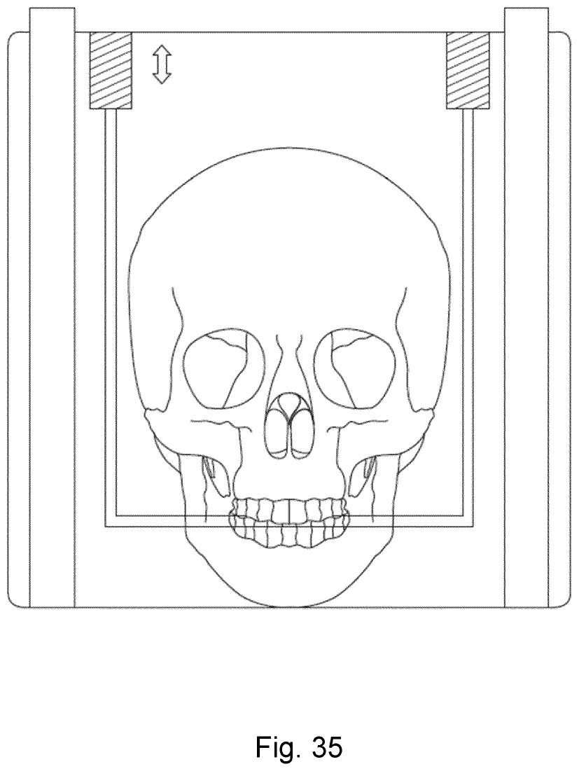

FIG. 35 illustrates a horizontal platform shaped for access to the mouth connected by two arms to actuators which may be mounted to the backboard or on rails such as in FIGS. 29, 30, 31 and 32. As the platform actuates it exerts a force to its anchor points in the mouth.

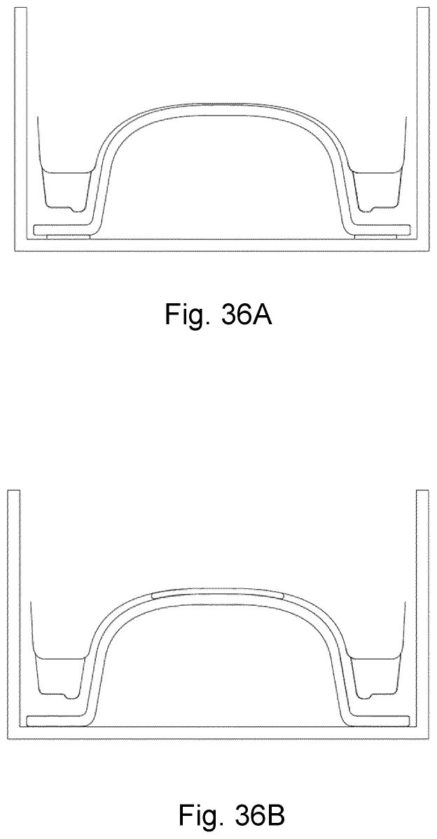

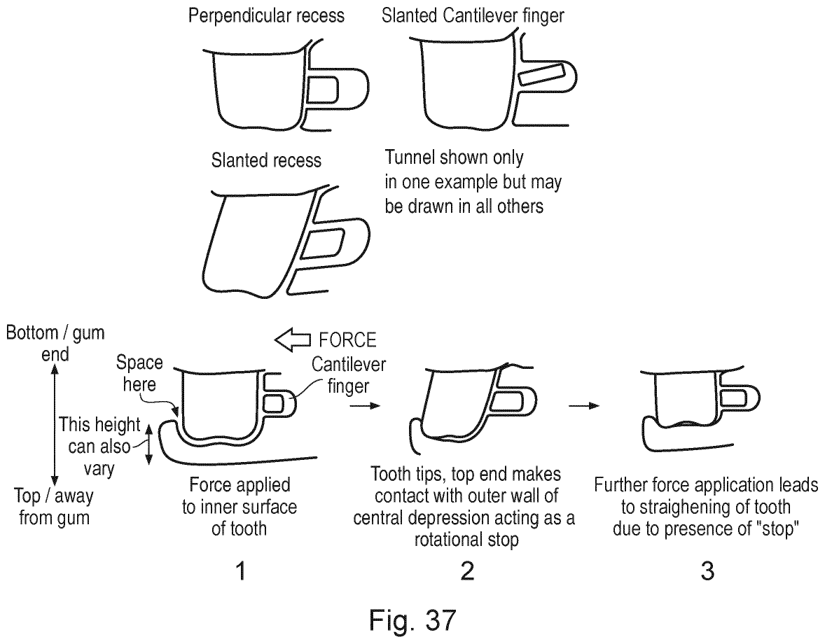

FIGS. 36A and 36B illustrate structures which attach to the horizontal platform and in these examples are arranged to apply force to the palate without involving the teeth. The inflatable structure are arranged between the lateral arms of the device and the horizontal platform, as shown in FIG. 36A. In the alternative arrangement shown in FIG. 36B, the inflatable structure is arranged between the palate and the curved surface of the device. In each of the arrangements shown in FIGS. 36A and 36B, the inflatable structures act to apply force to the palate. In FIG. 36A the force is applied through actuation of the device, and in the case of the arrangement shown in FIG. 36B, the force is applied directly to the palate. In each of these arrangements, the horizontal platform is fixed in position either applying a constant force indirectly (i.e. through the intra-oral device) or not at all. The inflatable cushions can be also used as ‘active cushioning’ while the horizontal platform is actuated. For example, the inflatable structures can be sealed so that they can passively absorb the force which is acted upon them by the platform. Alternatively, the inflatable structures may be connected to a hydraulic actuator which is configured to cause application of either a constant or dynamic force.

According to an alternative arrangement to that which is shown in FIGS. 36A and 36B, inflatable structures may be arranged between the arms of the device and the occlusal plane of the teeth. Thus, activation of these inflatable structures would cause a direct application of force upon the teeth. Such an arrangement of inflatable structures may be provided in addition to either of the arrangements shown in FIGS. 36A and 36B.

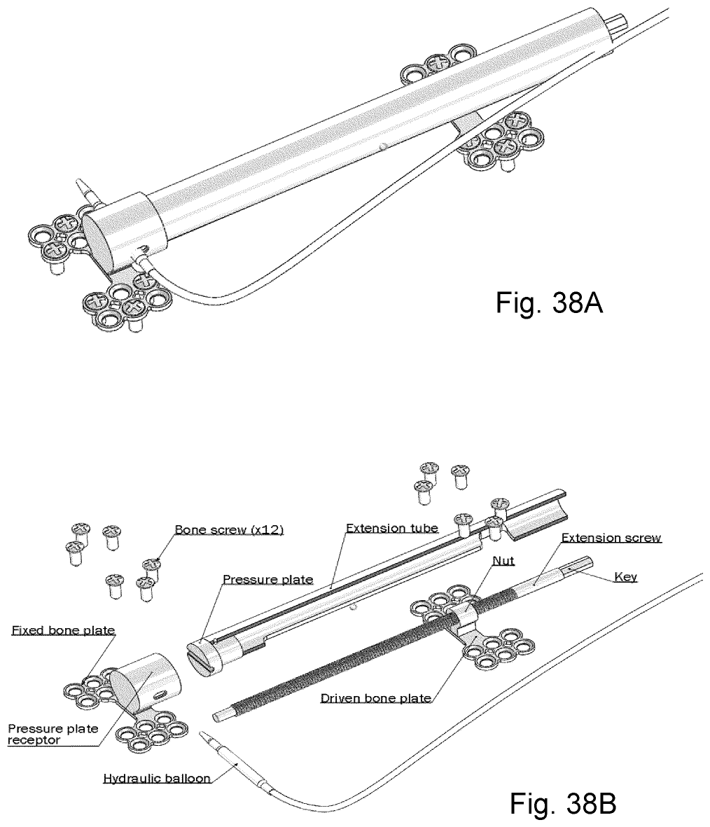

FIGS. 38A and 38B show a Matthew-Tessiers distractor which may be used in combination with embodiments of the present invention.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the scope of the invention. All references referred to above are hereby incorporated by reference.

1. A device for cranial restructuring, by the expansion, compression, or flexure of a cranial structure located between a first anchor point and a second anchor point, the device including:
a force generator configured to generate a force;
a first anchor for attachment to the first anchor point;
a second anchor for attachment to a second anchor point;
a hydraulic force transmitting structure, connected to the force generator, and configured to transmit the force to the first anchor and the second anchor thereby putting the cranial structure in at least one of: tension, compression or flexure, wherein the force transmitting structure is a hydraulic force transmitting structure.

2. A device according to claim 1, wherein:
the generated force includes a periodic component.

3. A device according to claim 1, wherein:
the generated force is constant or substantially constant.

4. A device according to claim 1, wherein:
the force transmitting structure includes an inflatable structure.

5. A device according to claim 4, wherein:
the force transmitting structure includes a first inflatable structure rigidly connected to the first anchor, and a second inflatable structure rigidly connected to the second anchor.

6. A device according to claim 5, wherein:
the first inflatable structure and the second inflatable structure are connected in series with each other.

7. A device according to claim 4, wherein:
the force transmitting structure includes a first channel including the inflatable structure.

8. A device according to claim 7, wherein:
the force transmitting structure includes a moulded structure which includes a first recess which is shaped to conform to at least one of an inner, an outer and an occlusal surface of the first tooth, the recess including a first intermediate structure, a surface of which is arranged to contact a surface of the first tooth when the device is in place inside a user's mouth.

9. A device according to claim 8, wherein:
the first channel is located within the portion of the moulded structure which is behind the first intermediate structure, and the inflatable structure is arranged such that when it is inflated, it exerts a force on the first intermediate structure, and the force is transmitted to the first tooth via the first intermediate structure.

10. A device according to claim 9, wherein:
the moulded structure includes a plurality of recesses, each shaped to conform to an inner surface of a respective tooth, and each recess including a respective intermediate structure, a surface of which is arranged to contact a surface of the respective tooth when the device is in place inside a user's mouth.

11. A device according to claim 10, wherein:
the first channel is arcuate, is located behind each of the respective cantilever structures, such that when the inflatable structure is inflated, it exerts a force on each of the intermediate structures, and the force is transmitted to each of teeth via the respective intermediate structures.

12. A device according to claim 7, wherein the intermediate structure is at least one of a cantilever structure, a sliding member and a weakened portion of a wall of the recess.

13. A device according to claim 1 wherein:
the device is modular.

14. A device according to claim 1, wherein:
the force generator is removable.

15. A device according to claim 1 wherein:
the force generator is connectable to at least a portion of the force transmitting structure by a detachable self-sealing connection.

16. A device according to claim 1, wherein:
the detachable self-sealing connection includes a valve.

17. A device according to claim 1, wherein:
the detachable self-sealing connection includes at least one of a one-way valve and a reversible valve.

18. A device according to claim 1, wherein:
the force transmitting structure is configured to transmit a first force to the first anchor and a second force to the second anchor, wherein the first force is in the opposite direction from the second force.

19. A device according to claim 18, wherein:
the force transmitting structure is configured to apply the generated force directly to the first anchor only, and wherein in use, the first force is applied to the second anchor via the cranial structure, as a reaction force.

20. A device according to claim 1, wherein:
the force transmitting structure is configured to apply the generated force directly to the first anchor and the second anchor.

21. A device according to claim 1, wherein the device defines an expansion mechanism for performing maxillary or mandibular expansion,
the hydraulic force transmitting structure comprises a first and a second hydraulic component, a first and a second channel component arranged to house, respectively, the first and second hydraulic components, and a central component arranged to couple the first and second channel components;
the first anchor point defines an attachment component of the first channel component and the second anchor point defines an attachment component of the second channel component.

22. A device according to claim 21, wherein the attachment component comprises a wing portion which extends from a main body of the attachment component, the wing portion comprises a hole configured to receive a fastener which is configured to fasten the attachment means to a cranial structure of the user.

23. A device according to claim 22, wherein the hole comprises a locating slot which is configured to releasably couple the wing portion to the fastener, wherein the locating slot comprises two intersecting circular holes, a first hole being larger than a diameter of a head portion of the fastener and a second smaller hole being larger than a diameter of a shaft portion of the fastener and smaller than the diameter of the head portion of the fastener.

24. A device for cranial restructuring, by the expansion, compression, or flexure of a cranial structure having a first anchor point, the device including:
a head support, having a head-receiving portion which is configured to receive a portion of a user's head;
a force generator configured to generate force which is periodic;
a first anchor for attachment to the first anchor point;
a force transmitting structure, connected to the force generator, and configured to transmit the periodic force to the first anchor in a restructuring direction;
wherein the head support includes:
a restriction means configured, in use, to prevent or restrict movement of the user's head in the restructuring direction, when the periodic force is being applied.

25. A device according to claim 24, wherein:
the force transmitting structure is a hydraulic force transmitting structure.

26. A device for cranial restructuring, by the expansion, compression, or flexure of a cranial structure having a first anchor point, the device including:
a head support, having a head-receiving portion which is configured to receive a portion of a user's head;
a force generator configured to generate a force;
a first anchor for attachment to the first anchor point;
a force transmitting structure, connected to the force generator, and configured to transmit the periodic force to the first anchor in a restructuring direction, wherein the force transmitting structure is a hydraulic force transmitting structure;
wherein the head support includes:
a restriction means configured, in use, to prevent or restrict movement of the user's head in the restructuring direction, when the periodic force is being applied.

27. A device according to claim 26, wherein:
the generated force is constant or substantially constant.

28. A device according to claim 26, wherein:
the generated force includes a periodic component.

29. A device according to claim 24, wherein:
first anchor is a screw, configured to contact bone or soft tissue;
the force transmitting structure includes one or more wires or rods connecting the force generator to the screw.

30. A device according to claim 24, wherein:
the head support is configured to be tightened around a user's head such that force is exerted against the sides, front or back of the user's head and/or face, such that the inner surfaces of the head support form at least part of the restriction means, restricting movement of the user's head by friction.

31. A device according to claim 24, wherein:
the restriction means includes an abutment surface configured to abut the user's head in use, wherein contact with the abutment surface is configured to prevent or restrict movement of the user's head in the restructuring direction.

32. A device according to claim 24, wherein:
the device includes a cradle having a rail.

33. A device according to claim 32, wherein:
the rail is connected to the head support by one or more connectors.

34. A device according to claim 33, wherein:
the device includes a first connector and a second connector, the rail being attached to an end of the connectors which is distal to the force generator.

35. A device according to claim 34, wherein:
the device further includes a third connector, which is connected to the rail at a proximal end, and the first anchor at a distal end.

36. A device according to claim 24, wherein:
the device further includes a second anchor for connection to a second anchor point of the cranial structure.

37. A device according to claim 36, wherein:
the device further includes a fourth connector, which is connected to the rail at a proximal end, and the first anchor at a distal end.

38. A device according to claim 24, wherein:
the device includes a plurality of rails.

39. A device according to claim 38, wherein:
each rail of the plurality of rails is connected to at least one force generator, by at least one connector.

40. A device according to claim 24, wherein:
the restriction means is located on a rail.

41. A device according to claim 24, wherein:
the force generator includes one or more of a motor and a hydraulic pump.

42. A device according to claim 1, wherein:
the first anchor point is a first tooth, and the first anchor includes a first tooth-contacting component which is configured either to:
wrap around the first tooth, or
abut an inner surface or outer surface of the first tooth.

43. A device according to claim 42, wherein:
the first tooth-contacting component includes one or more of: a wire, a band, a plate, or another orthodontic fixation.

44. A device according to claim 42, wherein:
the second anchor point is a second tooth, and the second anchor includes a second tooth-contacting component which is configured either to:
wrap around the second tooth, or
abut an inner surface or outer surface of the second tooth.

45. A device according to claim 44, wherein:
the second tooth-contacting component includes one or more of: a wire, a band, a plate, or another orthodontic fixation.

46. A device according to claim 42, wherein:
the first tooth-contacting component or the second tooth-contacting component includes a portion which is shaped to conform to a surface of the first tooth or second tooth, respectively.

47. A device according to claim 1, wherein:
one or both of the first anchor and the second anchor includes a screw which is configured to contact bone or soft tissue directly.

48-55. (canceled)

56. A device according to claim 1, wherein:
the force generator is configured to generate a force having a profile including non-periodic regions and periodic regions.

57. A device according to claim 1, wherein:
the force generator is configured to receive input from a measurement device, and to adjust the characteristics of the generated force in response to the input from the measurement device.

58. A device according to claim 1, wherein:
the force generator is configured to adjust the characteristics of the force to maintain a constant rate of restructuring of the cranial structure.

59. A device according to claim 1, wherein:
the measurement device includes: a pressure gauge, a strain gauge, a device for measuring displacement of the cranial structure, an electrocardiogram machine, an electroencephalogram machine, or an electromyography machine.

60. A device according to claim 1, wherein:
the device includes a first component configured to deliver a static force to the first anchor and/or second anchor, and a second component configured to deliver the periodic force to the first anchor and/or second anchor.

61. A device according to claim 1, where:
the first component is adjustable.

62. A device according to claim 1, wherein:
the force generator is an external force generator.

63. A device according to claim 1, wherein:
the force transmitting structure is configured to transmit force from outside a user's body to a location inside the user's body.

64. A device according to claim 1, wherein:
the force generator is located within a housing which is configured to attachment to a harness.

65. A device according to claim 1, wherein:
the force transmitting structure includes one or more inextensible, incompressible wires configured to transmit the force as tension.
